SEQUENCE LISTING (5 sequences)

SEQ ID NO: 1 - DNA, 31 bp, Artificial Sequence
  Probe
ccttccttatcctggatcttggcaaaatcga

SEQ ID NO: 2 - DNA, 31 bp, Artificial Sequence
  Probe
tcgattttctttacattttctatcgtatccg

SEQ ID NO: 3 - DNA, 167 bp, Artificial Sequence
  Probe
gtaaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaa

SEQ ID NO: 4 - DNA, 37 bp, Artificial Sequence
  Probe
atcctggatcttggacaaggaggggaactgatcccct

SEQ ID NO: 5 - DNA, 19 bp, Artificial Sequence
  Probe
ccttctttacattttctat

(19) United States
(12) Patent Application Publication
(10) Pub. No.: US 2004/0067498 A1
(43) Pub. Date: Apr. 8, 2004

(54) Detection of nucleic acid sequences by cleavage and separation of tag-containing structures

(76) Inventors: Ahmed Chenna, Sunnyvale, CA (US); Vivian Xiao, Cupertino, CA (US); Sharat Singh, San Jose, CA (US)

Correspondence Address: ACLARA BIOSCIENCES, INC., 1288 PEAR AVENUE, MOUNTAIN VIEW, CA, 94043, US

(21) Appl. No.: 10/289,309

(22) Filed: Nov. 6, 2002

Related U.S. Application Data
(63) Continuation-in-part of application No. 09/602,586, filed on Jun. 21, 2000, now Pat. No. 6,514,700.
(63) Continuation-in-part of application No. 09/561,579, filed on Apr. 28, 2000, now Pat. No. 6,682,887.
(60) Provisional application No. 60/337,686, filed on Nov. 9, 2001.

(51) Int. Cl. C12Q 1/68

(52) U.S. Cl. 435/6

(57) ABSTRACT

The present invention is directed to a method of detecting pluralities of target nucleic acid sequences by forming and cleaving duplex structures with a pair of probes, one probe of each pair being labeled with an electrophoretic tag. Cleavage of the duplex structures releases electrophoretic tags that are then separated and identified to indicate the presence or quantity of the target sequences. The present invention is particularly useful in multiplex reactions wherein multiple target sequences are detected in one reaction. Kits useful in the detection of nucleic acids are also provided.

This application is a continuation-in-part of co-pending application Ser. No. 09/602,586 filed 21 Jun. 2000 and co-pending application Ser. No. 09/561,579 filed 28 Apr. 2000. This application further claim priority from provisional application Ser. No. 60/337,686 filed 9 Nov. 2001. All of the above-identified co-pending applications are hereby incorporated by reference.

### FIELD OF THE INVENTION

This invention relates to methods of detecting and/or quantitating nucleic acid sequences of interest. In particular, this invention is useful in the simultaneous detection or quantitation of a plurality of target nucleic acid sequences, especially selected pluralities of expressed genes.

### BACKGROUND OF THE INVENTION

The development of several powerful technologies for genome-wide expression measurements has created an opportunity to study and understand the coordinated activities of large sets of, if not all, an organism's genes in response to a wide range of conditions and stimuli, e.g. DeRisi et al, Science, 278: 680-686 (1997); Wodicka et al, Nature Biotechnology, 15: 1359-1367 (1997); Velculescu et al, Cell, 243-251 (1997); Brenner et al, Nature Biotechnology, 18: 630-634 (2000). Studies using these technologies have shown that reduced subsets of genes appear to be co-regulated to perform particular functions and that subsets of expressed genes can be used to classify cells phenotypically, e.g. Shiffman and Porter, Current Opinion in Biotechnology, 11: 598-601 (2000); Afshari et al, Nature, 403: 503-511 (2000); Golub et al, Science, 286: 531-537 (1999); van't Veer et al, Nature, 415: 530-536 (2002); and the like.

An area of interest in drug development is the expression profiles of genes involved with the metabolism or toxic effects of xenobiotic compounds. Several studies have shown that sets of several tens of genes can serve as indicators of compound toxicity, e.g. Thomas et al, Molecular Pharmacology, 60: 1189-1194 (2001); Waring et al, Toxicology Letters, 120: 359-368 (2001); Longueville et al, Biochem. Pharmacology, 64: 137-149 (2002); and the like.

Accordingly, there is an interest in technologies that provide convenient and accurate measurements of multiple expressed genes in a single assay. Current approaches to such measurements include multiplexed polymerase chain reaction (PCR), spotted and synthesized DNA microarrays, color-coded microbeads, and single-analyte assays used with robotics apparatus, e.g. Longueville et al (cited above); Elnifro et al, Clinical Microbiology Reviews, 13: 559-570 (2000); Chen et al, Genome Research, 10: 549-557 (2000); and the like. Unfortunately, none of the approaches provides a completely satisfactory solution for the desired measurements for several reasons including difficulty in automating, reagent usage, sensitivity, consistency of results, and so on, e.g. Elnifro et al (cited above); Hess et al, Trends in Biotechnology, 19: 463-468 (2001); King and Sinha, JAMA, 286: 2280-2288 (2001).

In view of the above, drug development and medical diagnostics would be advanced by the availability of a non-array-, non-PCR-based method for accurate, convenient, and simultaneous measurement of the expression of multiple genes in a single cellular or tissue sample.

### SUMMARY OF THE INVENTION

The present invention is directed to methods and compositions for detecting the presence or absence of a plurality of target polynucleotides in a sample by forming nucleic acid structures containing a site recognized by a cleaving agent. After such formation, the structures are selectively cleaved to release tags, which are then separated and identified. In one aspect, released tags accumulate because of the equilibrium exchange or cycling of probes between the bound state in a structure and a free state in solution. In another aspect, cleavage of the nucleic acid structure accelerates disassociation by destabilization of the structures. In both cases, dissociation frees the target polynucleotide for a new cycle of structure formation and cleavage, thereby permitting the accumulation of released tags.

In another aspect, the invention provides a method for detecting the presence or absence of a plurality of target polynucleotides in a sample comprising the following steps: (1) providing for each polynucleotide a helper probe complementary to a region of the polynucleotide and an electrophoretic probe complementary to the helper probe and to the polynucleotide adjacent to said region, such that the helper probe and the electrophoretic probe form a recognition duplex upon hybridization to each other and to the polynucleotide, each electrophoretic probe having attached an electrophoretic tag with a separation or detection characteristic distinct from those of other electrophoretic tags so that each electrophoretic tag forms a distinguishable peak in a separation profile; (2) combining under hybridization conditions the sample, the helper probes, and the electrophoretic probes to form an assay mixture such that recognition duplexes are formed; (3) cleaving the recognition duplexes at a cleavage site so that electrophoretic tags are released; and (4) separating and identifying the released electrophoretic tags to detect each of the plurality of polynucleotides.

In another aspect, the present invention includes kits for performing the methods of the invention, such kits comprising pairs of helper probes and electrophoretic probes for detecting or measuring the quantities of each of a plurality of predetermined target polynucleotides. Such kits further comprising a cleavage agent for cleaving the nucleic acid structures formed among the helper probes, electrophoretic probes, and target polynucleotides.

The present invention provides a detection and signal generation means with several advantages over presently available techniques for multiplexed measurements of target polynucleotides, including but not limited to the following: (1) detection and/or measurement of tags that are separated from the assay mixture provide greatly reduced background and a significant gain in sensitivity; (2) use of tags that are specially designed for ease of separation provides convenient multiplexing capability; (3) re-formation of nucleic acid structures after cleavage and tag release permit signal amplification; (4) the method is practiced under isothermal conditions, which eliminates the need of expensive thermal cycling equipment; (5) formation of a double stranded recognition structure between the helper probes and electrophoretic probes for cleavage provides for a wide selection of cleavage agents that selectively operate only on double stranded substrates.

### BRIEF DESCRIPTION OF THE DRAWINGS

FIG. 1A illustrates an example of how a helper probe and an electrophoretic probe combines with a target polynucleotide to form a stable complex containing a recognition duplex.

FIG. 1B illustrates the operation of one embodiment of the invention for detecting a plurality of target polynucleotides.

FIG. 2A illustrates an example of an assay in accordance with the invention in which the cleavage agent is hOGG1 protein.

FIG. 2B illustrates an example of an assay in accordance with the invention in which the cleavage agent is MutY protein.

FIG. 3A provides predicted and experimental (*) elution times of electrophoretic tags separated by capillary electrophoresis. C₃, C₆, C₉, and C₁₈ are commercially available phosphoramidite spacers from Glen Research, Sterling Va. The units are derivatives of N,N-diisopropyl, O-cyanoethyl phosphoramidite, which is indicated by “Q”. C₃ is DMT (dimethoxytrityl)oxypropyl Q; C₆ is DMToxyhexyl Q; C₉ is DMToxy(triethyleneoxy) Q; C₁₂ is DMToxydodecyl Q; C₁₈ is DMToxy(hexaethyleneoxy) Q.

FIGS. 3B-3I shows the structures of exemplary released electrophoretic tags.

FIG. 4 shows multiple electropherograms showing separation of individual electrophoretic tags. The figure illustrates obtainable resolution of the reporters, which are identified by their ACLA numbers.

FIG. 5 illustrates phosphoramidite precursors for synthesizing electrophoretic probes on a conventional DNA synthesizer.

FIG. 6 shows charge modifier phosphoramidites (EC or CE is cyanoethyl and DMT is dimethyltrityl).

FIG. 7 illustrates a scheme for producing a fluorescein phosphoramidite using a hydroxylamine precursor.

FIG. 8A illustrates one exemplary synthetic approach starting with commercially available NHS ester of 6-carboxy fluorescein, where the phenolic hydroxyl groups are protected using an anhydride. Upon standard extractive workup, a 95% yield of product is obtained. This material is phosphitylated to generate the phosphoramidite monomer.

FIG. 8B illustrates the use of a symmetrical bis-amino alcohol linker as the amino alcohol with the second amine then coupled with a multitude of carboxylic acid derivatives.

FIG. 9A illustrates the use of an alternative strategy that uses 5-aminofluorescein as starting material and the same series of steps to convert it to its protected phosphoramidite monomer.

FIG. 9B illustrates several separation modifiers that can be used for conversion of amino dyes into e-tag phosphoramidite monomers.

FIG. 10 gives the structure of several electrophoretic tags derived from maleimide-linked precursors.

FIG. 11 shows the projected result of an assay with or without the enzyme.

### DETAILED DESCRIPTION OF THE INVENTION

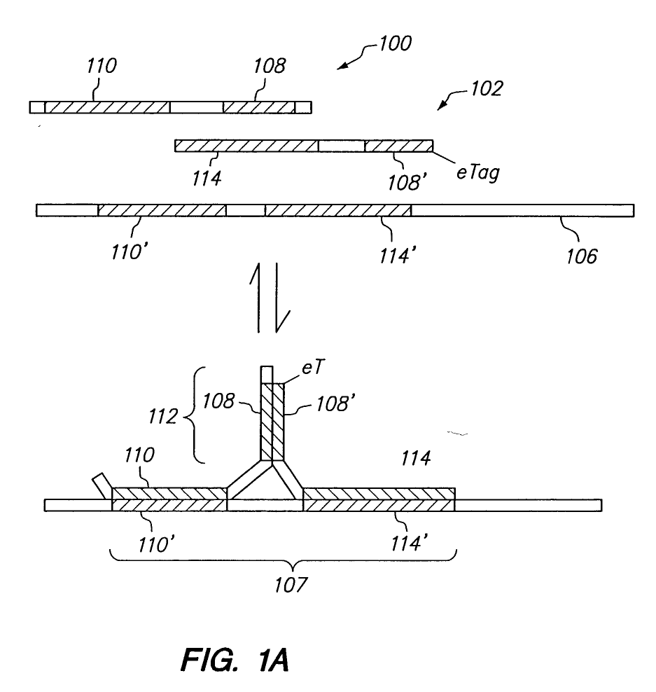

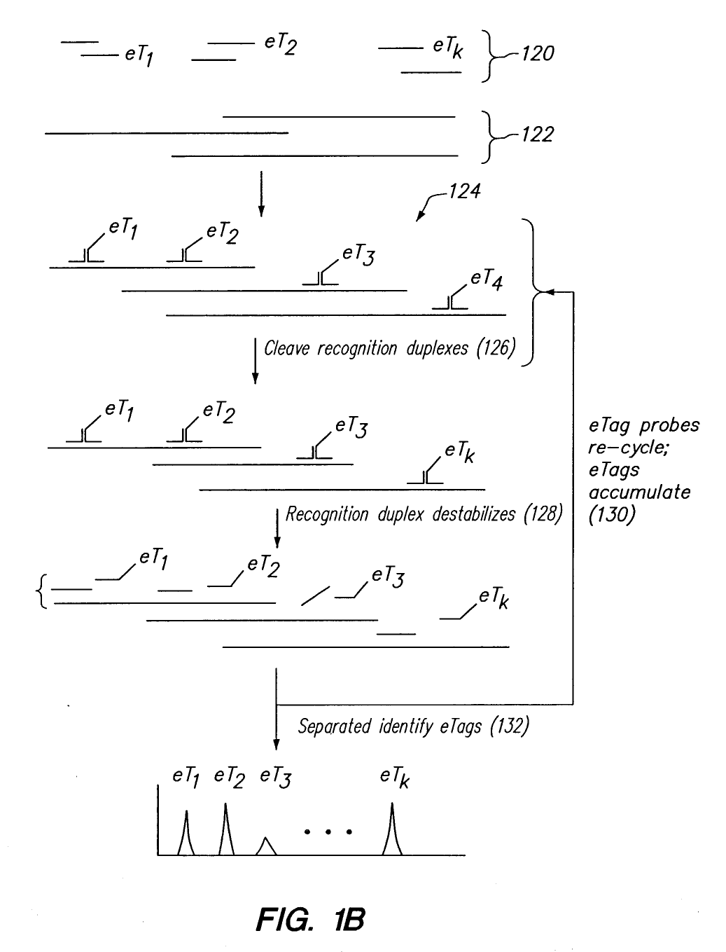

The present invention is directed to a method of detecting, and/or measuring the quantity of, a plurality of target polynucleotides in the same assay mixture. In accordance with the method of the invention, for each target polynucleotide to be detected, a helper probe and an electrophoretic probe are provided that have complementary regions with one another, but which are designed not to form stable duplexes with one another under assay conditions, unless a single stranded form of their corresponding target polynucleotide is present. Thus, as illustrated in FIG. 1A, a stable multi-strand complex is formed under assay conditions only in the presence of all three members: helper probe (100), electrophoretic probe (102), and target polynucleotide (106). As with the helper probe and the electrophoretic probe, electrophoretic probe (102) is designed so that alone it is unable to form a stable duplex with target polynucleotide (106) under predetermined assay conditions. When such a complex (107) is formed, the complementary regions of the helper probe (108) and electrophoretic probe (108′) hybridize to form a recognition duplex (112). In order to form a three-strand complex, both helper probe (100) and electrophoretic probe (102) have complementary regions (110) and (114) to sites (110′) and (114′), respectively, of target polynucleotide (106). Target polynucleotide (106) may be either a single stranded DNA or a single stranded RNA, such as a messenger RNA (mRNA).

As illustrated in FIG. 1B, in the operation of an assay of the invention, pairs (120) of helper probes and electrophoretic probes are combined with a plurality of target polynucleotides (122) under conditions that permit the formation of recognition duplexes (124) among the pairs whenever their corresponding target polynucleotide is present. Recognition duplexes (124) are recognized by a cleavage agent that specifically cleaves (126) only nucleic acids that are present in duplex form to release a fragment of the electrophoretic probe that is referred to herein as an electrophoretic tag, or “eTag.” Thus, single stranded nucleic acids, including unbound helper probe, unbound electrophoretic probe, and target polynucleotides are not cleaved or modified. Preferably, the cleavage agent is a nuclease whose substrate is, or includes, a duplex structure comprising two DNA strands, two RNA strands, or a DNA strand and an RNA strand. After cleavage of the electrophoretic probe, the recognition duplex de-stabilizes because fewer nucleotides are based-paired in the duplex, which, in turn, leads to the destabilization (128) of the entire three-strand complex. Under the assay conditions, which include providing the electrophoretic probe in substantial excess concentration over the target polynucleotides, uncleaved electrophoretic probe participates in successive cycles (130) of complex formation and cleavage until a detectable quantity of released electrophoretic tags accumulate in the assay mixture. After the assay reaction is complete, release electrophoretic tags are separated and identified (132) using conventional separation techniques, e.g. capillary electrophoresis, microbore chromatography, or the like.

As described more fully below, an important aspect of the invention is the set of electrophoretic tags generated in an assay. Generally, a set of electrophetic tags may be selected from a group of molecules having a wide variety of structures. The primary criterion for constructing a set is that each electrophoretic tag must be distinguishable from all the other electrophoretic tags of the same set under a predetermined method of separation and detection, as described in Singh, U.S. Pat. No. 6,322,980; Singh, PCT publication WO 00/66607; and Singh et al, PCT publication WO 01/83502, which references are incorporated by reference. That is, each electrophoretic tag of a set must have distinct detection and/or separation characteristics that allow it to be detected and quantified after separation with the other tags. Preferably, electrophoretic tags are detected by fluorescence characteristics and separated by electrophoresis; however, other liquid phase separation techniques, especially chromatography, may also be used. Electrophoretic tags of a set may be selected empirically; however, as illustrated below, members of a set may also be assembled from molecular building blocks with predictable separation characteristics.

Samples containing target polynucleotides may come from a wide variety of sources including cell cultures, animal or plant tissues, microorganisms, or the like. Samples are prepared for assays of the invention using conventional techniques, which may depend on the source from which a sample is taken. Guidance for sample preparation techniques can be found in standard treatises, such as Sambrook et al, Molecular Cloning, Second Edition (Cold Spring Harbor Laboratory Press, New York, 1989); Innis et al, editors, PCR Protocols (Academic Press, New York, 1990); Berger and Kimmel, “Guide to Molecular Cloning Techniques,” Vol. 152, Methods in Enzymology (Academic Press, New York, 1987); or the like. For mammalian tissue culture cells, or like sources, samples of target RNA may be prepared by conventional cell lysis techniques (e.g. 0.14 M NaCl, 1.5 mM MgCl2, 10 mM Tris-Cl (pH 8.6), 0.5% Nonidet P-40, 1 mM dithiothreitol, 1000 units/mL placential RNAase inhibitor or 20 mM vanadyl-ribonucleoside complexes).

Definitions

“Capillary electrophoresis” means electrophoresis in a capillary tube or in a capillary plate, where the diameter of the separation column or thickness of the separation plate is between about 25-500 microns, allowing efficient heat dissipation throughout the separation medium, with consequently low thermal convection within the medium.

A “sieving matrix” or “sieving medium” means an electrophoresis medium that contains crosslinked or non-crosslinked polymers which are effective to retard electrophoretic migration of charged species through the matrix.

“Specific” or “specificity” in reference to the binding of one molecule to another molecule, such as a probe for a target polynucleotide, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough so that short range non-covalent chemical interactions, such as Van der Waal forces, hydrogen bonding, hydrophobic interactions, and the like, dominate the interaction of the molecules.

As used herein, the term “spectrally resolvable” in reference to a plurality of fluorescent labels means that the fluorescent emission bands of the labels are sufficiently distinct, i.e. sufficiently non-overlapping, that electrophoretic tags to which the respective labels are attached can be distinguished on the basis of the fluorescent signal generated by the respective labels by standard photodetection systems, e.g. employing a system of band pass filters and photomultiplier tubes, or the like, as exemplified by the systems described in U.S. Pat. Nos. 4,230,558, 4,811,218, or the like, or in Wheeless et al, pgs. 21-76, in Flow Cytometry: Instrumentation and Data Analysis (Academic Press, New York, 1985).

“Oligonucleotide” as used herein means linear oligomers of natural or modified nucleosidic monomers linked by phosphodiester bonds or analogs thereof. Oligonucleotides include deoxyribonucleosides, ribonucleosides, anomeric forms thereof, peptide nucleic acids (PNAs), and the like, capable of specifically binding to a target polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g. 3-4, to several tens of monomeric units, e.g. 40-60. Whenever an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides of the invention comprise the four natural deoxynucleotides; however, they may also comprise ribonucleosides or non-natural nucleotide analogs. It is clear to those skilled in the art when oligonucleotides having natural or non-natural nucleotides may be employed in the invention. For example, where processing by an enzyme is called for, usually oligonucleotides consisting of natural nucleotides are required. Likewise, where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

The term “polynucleotide” means a linear polymer of natural nucleotides, including deoxynucleotides and ribonucleotides. Polynucleotides include but are not limited to DNA, RNA, messenger RNA, cloning vectors, expression vectors, transposons, genomic DNA, plasmids, cosmids, phages, viruses, bacterial genomes, and like compounds.

“Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding. As used herein, “stable duplex” between complementary oligonucleotides or polynucleotides means that a significant fraction of such compounds are in duplex or double stranded form with one another as opposed to single stranded form. Preferably, such significant fraction is at least ten percent of the strand in lower concentration, and more preferably, thirty percent.

As used herein, “nucleoside” includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the only proviso that they are capable of specific hybridization. Such analogs include synthetic nucleosides designed to enhance binding properties, reduce complexity, increase specificity, and the like.

As used herein, “probe” may refer to “helper probe” and/or “electrophoretic probe” either each alone or both collectively depending on context.

As used herein, “amplicon” means the product of an amplification reaction. That is, it is a population of polynucleotides, usually double stranded, that are replicated from one or more starting sequences. The one or more starting sequences may be one or more copies of the same sequence, or it may be a mixture of different sequences. Preferably, amplicons are produced either in a polymerase chain reaction (PCR) or by replication in a cloning vector.

A “target sequence” or “target polynucleotide” is a nucleic acid sequence of interest to be detected or quantitated.

A probe is “capable of hybridizing” to a nucleic acid sequence if at least one region of the probe shares substantial sequence identity with at least one region of the complement of the nucleic acid sequence. “Substantial sequence identity” is a sequence identity of at least about 80%, preferably at least about 85%, more preferably at least about 90%, and most preferably 100%. It should be noted that for the purpose of determining sequence identity of a DNA sequence and a RNA sequence, U and T are considered the same nucleotide. For example, a probe comprising the sequence ATCAGC is capable of hybridizing to a target RNA sequence comprising the sequence GCUGAU.

It is contemplated that a probe of the invention may comprise additional nucleic acid sequences that do not share any sequence identity with the target sequence. Conversely, it is also contemplated that the target sequence comprises additional nucleic acid sequences that do not share any sequence identity with the probe. Preferably, the probe and the target sequence share substantial sequence identity in a region of at least about 6 consecutive nucleotides. The region of substantial sequence identity is more preferably at least about 8 consecutive nucleotides, yet more preferably at least about 10 consecutive nucleotides, and most more preferably at least about 12 consecutive nucleotides.

As used herein, the term “Tm” is used in reference to the “melting temperature.” The melting temperature is the temperature at which a population of double-stranded nucleic acid molecules becomes half dissociated into single strands. Several equations for calculating the Tm of nucleic acids are well known in the art. As indicated by standard references, a simple estimate of the T, value may be calculated by the equation. Tm=81.5+0.4 1 (% G+C), when a nucleic acid is in aqueous solution at I M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G.T mismatches in DNA. Biochemistry 36, 10581-94 (1997) include more sophisticated computations which take structural and environmental, as well as sequence characteristics into account for the calculation of Tm.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

The term “source” in reference to target polynucleotide means any sample that contains polynucleotides (RNA or DNA). Particularly preferred sources of target nucleic acids are biological samples including, but not limited to cultures, blood, saliva, cerebral spinal fluid, pleural fluid, milk, lymph, sputum, semen, and animal or plant tissue.

The term “isothermal” in reference to assay conditions means a uniform or constant temperature at which the cleavage of the electrophoretic probe in accordance with the present invention is carried out. The temperature is chosen so that the duplex formed by hybridizing the probes to a polynucleotide with a target polynucleotide sequence is in equilibrium with the free or unhybridized probes and free or unhybridized target polynucleotide sequence, a condition that is otherwise referred to herein as “reversibly hybridizing” the probe with a polynucleotide. Normally, at least 1%, preferably 20 to 80%, usually less than 95% of the polynucleotide is hybridized to the probe under the isothermal conditions. Accordingly, under isothermal conditions there are molecules of polynucleotide that are hybridized with the probes, or portions thereof, and are in dynamic equilibrium with molecules that are not hybridized with the probes. Some fluctuation of the temperature may occur and still achieve the benefits of the present invention. The fluctuation generally is not necessary for carrying out the methods of the present invention and usually offer no substantial improvement. Accordingly, the term “isothermal” includes the use of a fluctuating temperature, particularly random or uncontrolled fluctuations in temperature, but specifically excludes the type of fluctuation in temperature referred to as thermal cycling, which is employed in some known amplification procedures, e.g., polymerase chain reaction.

As used herein, the term “kit” refers to any delivery system for delivering materials. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

Formation and Cleavage of Recognition Duplexes

Preferably, the helper probe and electrophoretic probe comprise synthetic oligonucleotides produced using conventional techniques. As explained more fully below, the mobility-modifying region and detectable label of electrophoretic probes are preferably attached to the oligonucleotide portion by forming a phosphoramidite precursor that may be coupled to oligonucleotide portion in the final step of a probe's synthesis.

The helper probe and electrophoretic probe of each pair of such probes each possesses a region that hybridizes to a target polynucleotide and a region that hybridizes to the other probe of the pair to form a recognition duplex. The regions hybridizing to one another to form a recognition duplex have nucleotide sequences that are complementary to one another. This complementarity need not result in a perfectly matched duplex. Indeed, as described below, in some cases, the recognition duplex intentionally contains a mismatched basepair which serves as a specific recognition structure for a cleavage agent. These regions of the probe pairs are designed such that the melting temperature of the recognition duplex ins the absence of a target polynucleotide is less than the operating temperature of the assay, preferably 4° C. less (more preferably 7-10° C. less) than the operating temperature, so that little or no hybridization of the regions forming the recognition duplex occurs in the absence of target polynucleotide. Melting temperature, Tm, is defined as the temperature at which 50% of a given nucleic acid duplex has melted (i.e., has become single-stranded). The Tm is dependent on reaction conditions such as the salt concentration of the solution. The desired Tm is typically achieved by manipulation of the length and nucleotide base composition of the complementary regions. Other methods can also be utilized to adjust duplex Tm, including but not limited to incorporation of mismatches, replacement of some or all of the complementary basepairs with stability enhancing nucleotides or internucleotide linkages, e.g. peptide nucleic acids, phosphoramidates, 2′-methoxyribonucleosides, and the like. When the operating temperature of an assay reaction is about 60° C., the preferred length of exactly complementary regions forming a recognition duplex is approximately 8 to 20 contiguous bases (dependent on base composition and sequence). Other reaction conditions would potentially lead to a different size range; this is readily determined empirically.

Upon contacting the probes with a solution containing a target nucleic acid, the probe regions of the two probe oligonucleotides will hybridize to their respective target regions, which are typically adjacent to one another, as shown in FIG. 1 (they do not have to be immediately adjacent). When this occurs, the mutually complementary regions of the two probe strands are constrained to be in close proximity to one another, thus increasing the stability of the associated duplex. The regions forming the recognition duplex are designed such that the Tm of the duplex formed in the presence of target is approximately equal to or above the operating temperature of the assay, preferably 4° C. above (more preferably 7° C. or 10° C.) the operating temperature such that the mutually complementary regions will form a duplex. The preferred length of the mutually complementary regions of the probes is approximately 8 to 20 contiguous complementary bases (dependent on base composition and sequence). The regions of the probes that are complementary to a target polynucleotide can be designed in a variety of manners. For example, these regions can be designed similarly to the regions forming the recognition duplex in that the Tm of either region alone (i.e., one probe strand plus the target strand) is below the operating temperature, but is above the operating temperature when both probe strands and the target strand are present and the regions forming the recognition duplex are hybridized. They can also be designed such that the Tm's of the probe regions are both above the operating temperature, or they can be designed such that one Tm is above and one Tm is below the operating temperature. Whatever design is chosen, the requirement that the regions making up the recognition duplex form a stable duplex only in the presence of target must be met. The regions of the probes complementary to target polynucleotides are preferably between 8 and 50 nucleotides in length, more preferably between 8 and 30 nucleotides in length. These regions can be longer, but most applications do not require this additional length, and synthesis of these longer oligonucleotides is more costly and time consuming than the shorter oligonucleotides.

One or both probe sequences is chosen to react with the desired target polynucleotide(s) and preferably to not react with any undesired target polynucleotide(s) (i.e., cross-react). If one probe region hybridizes with an undesired target but the other probe region does not, the assay will still function properly since both probe segments have to hybridize in order for the recognition duplex to be formed.

To detect target polynucleotides in a sample using pairs of helper and electrophoretic probes described above, the following general procedure is used: 1) add the pairs of helper and electrophoretic probes to the sample, 2) incubate to allow annealing of the appropriate regions to occur, 3) cleave the recognition duplexes that form to release electrophoretic tags, and 4) separate and identify the released electrophoretic tags. The annealing conditions can be varied depending on the exact application, the design of the probe, the nature of the polynucleotide and the composition of the sample in which the target is contained. The conditions must be chosen, however, to fulfill the Tm requirements stated above. Preferably, the incubation temperature is preferably between 5° and 70° C., more preferably between 30° and 65° C.

Guidance for selecting assay conditions and oligonucleotide sequences for forming the above complexes between helper probes, electrophoretic probes, and target polynucleotides can be found in the art, e.g. Hogan et al, U.S. Pat. No. 5,451,503; Western et al, U.S. Pat. No. 6,121,001; Reynaldo et al, J. Mol. Biol., 297: 511-520 (2000); Wetmur, Critical Rev. in Biochem. Mol. Biol., 26: 227-259 (1991); and the like. Accordingly, these references are hereby incorporated by reference.

Recogniton duplexes are cleaved by a cleavage agent comprising either a chemical or a protein nuclease that requires a double stranded structure for cleavage to occur. A wide varity of cleavage agents may be used with the method of the invention. Chemical nucleases are described in the following references: Sigman et al, “Chemical nucleases: new reagents in molecular biology,” Annu. Rev. Biochem., 59: 207-236 (1990); and Thuong et al, “Sequence-specific recognition and modification of double-helical DNA by oligonucleotides,” Angew. Chem. Int. Ed. Engl., 32: 666-690 (1993). Generally, the oligonucleotide-based chemical nucleases have three components: i) an oligonucleotide moiety for sequence-specific binding, ii) a cleavage moiety, and iii) a linking moiety for attaching the oligonucleotide to the cleavage moiety. Sequence specific binding has been achieved by the formation of a Watson-Crick duplex with a single stranded target, by the formation of a “D-loop” with a double stranded target, and by the formation of a triplex structure with a double stranded target. In all of these cases, the oligonucleotide moiety defines the recognition site of the chemical nuclease.

The cleavage moiety may linked to the 5′ end, the 3′ end, to both ends, or to internal bases of the oligonucleotide moiety; thus, for oligonucleotide-based chemical nucleases, the recognition site may be separate from its cleavage site(s). The cleavage moieties for DNA targets typically are one of two types: a chemically activated agent for generating a diffusable radical, e.g. hydroxyl, that effects cleavage, or a tethered protein nuclease.

Preferably, recognition duplexes are cleaved with a protein nuclease that has well defined and repeatable cleavage properties. Suitable nucleases for use with the invention include, but are not limited to, restriction endonucleases and repair enzymes. Suitable nucleases for use with the invention include Fpg protein, endonuclease III (Nth) protein, AlkA protein, Tag protein, MPG protein, uracil-DNA glycosylase (UDG protein), MutY protein, T4 endonuclease V, cv-PDG protein, 8-oxo-guanine DNA glycosylase (hOGG1), FEN-1, human AP endonuclease, lambda exonuclease, RNase H, and the like. Such enzymes are commercially available from multiple vendors, New England Biolabs (Beverly, Mass.) and Trevigen Corp. (Gaithersburg, Md.). Many restriction endonucleases are suitable for use with the invention. Restriction endonucleases that can efficiently cleave at the end of a duplex are preferred so that released electrophoretic tags contains as few nucleotides as possible from the recognition duplex. Preferred restriction endonucleases include Tsp509 I, Nla III, BssK1, Dpn II, Mbo I, Sau 3A I, Mbo II, Ple I, Mnl I, Alw I, and the like, which are available from New England Biolabs (Beverly, Mass.). Preferably, thermal stable variants of nucleases are employed so that assay reaction temperature can be conducted in the range of from 40° C. to 70° C., and more preferably, in the range of from 40° C. to 65° C., and still more preferably, in the range of from 50° C. to 65° C.

A “DNA repair enzyme” is an enzyme that is a component of a DNA repair machinery, which enzyme is not a DNA polymerase. DNA repair enzymes include, for example, the enzymes participating in base excision repair (BER), nucleotide excision repair (NER) and mismatch repair (MMR). For a review of the role of chemical structure in determination of repair enzyme substrate specificity and mechanism, see Singer and Hang, Chapter 2, DNA and Free Radicals: Techniques, Mechanisms & Applications (Aruoma and Halliwell ed.), OICA International, 1998.

The base excision repair (BER) enzymes excise free bases from damaged DNA. The substrates for BER enzymes are mainly small DNA lesions such as oxidatively damaged bases, alkylation adducts, deamination products and certain types of single base mismatches. Base excision repair enzymes include DNA glycosylases such as Fpg protein, Nth protein, AlkA protein, Tag protein, MPG protein, UDG protein, Mut Y protein, T4 endonuclease V, and cv-PDG. These specific enzymes act at the first step of the BER pathway, in which DNA glycosylase hydrolyses the N-glycosylic bond connecting the altered base and the sugar-phosphate backbone, releasing a free base. The remaining abasic (AP) site is nicked by an AP endonuclease. Some glycosylases have associated AP lyase activity, which creates strand breaks 3′ to an AP site. Fpg and NTh proteins are DNA glycosylases/AP lyases recognizing and excising major purine and pyrimidine products of oxidative damage to DNA, respectively. AlkA protein removes a variety of damaged bases induced by alkylation, deamination or oxidation. Tag protein is a DNA glycosylase excising 3-methyladenosine and 3-methylguanine. These enzymes are active on damages present in double stranded DNA substrates. UDG (uracil-DNA glycosylase) removes uracil from both double and single-stranded DNA. MutY protein is a DNA glycosylase/AP lyase which recognizes adenine-guanine or adenine-cytosine mismatches and excises adenine. All of the above enzymes are of E. Coli origin.

In addition, human MPG (methylpurine glycosylase) recognizes alkylation, deamination, and oxidatively damaged bases in double stranded DNA. T4 endonuclease V is a glycosylase/AP lyase that is specific for UV light-induced cis-syn cyclobutane pyrimidine dimer (CPDs). Chlorella virus pyrimidine dimer glycosylase (cv-PDG) is specific not only for the cis-syn CPDs, but also for the trans-syn-II isomers. Typical glycosylases/lyases are listed in Table 1.

TABLE 1
Glycosylases

| | | | AP Lyase |
|---|---|---|---|
| Enzyme | Synonyms | Substrates | Activity |
| Fpg protein | E. coli Fapy- | 8-oxoguanine and | + |
| | DNA | formamidopyrimidines | |
| | glycosylase, | (FAPY-adenine, FAPY- | |
| | 8-oxoguanine | guanine), N⁷ or C⁸ alkylated | |
| | DNA | guanines modified by ring | |
| | glycosylase | opening, 5-hydroxy- | |
| | | cytosine, 5-hydroxyuracil | |
| Nth protein | E. coli | 5,6-dihydrothymine, 5- | + |
| | Endonuclease | hydroxy-5-methyl- | |
| | III, thymine | hydantoin, 5-hydroxy- | |
| | glycol-DNA | 6-uracil, alloxan, 5- | |
| | glycosylase | hydroxy-6-hydrouracil, | |
| | | thymine glycol, cytosine | |
| | | glycol, urea residues, | |
| | | pyrimidine hydrates, 5- | |
| | | hydroxycytosine. | |
| AlkA protein | E. coli 3- | 3-alkyladenine, 7- | − |
| | methyladenine- | alkylguanine, 3-alkyl- | |
| | DNA | guanine, O²-alkyl- | |
| | glycosylase II | pyrimidines, formyl | |
| | | uracil, hypoxanthine, | |
| | | hydroxymethyl uracil, | |
| | | adenine and guanine | |
| Tag protein | E. coli 3- | 3-methyladenine and 3- | − |
| | methyladenine- | methylguanine | |
| | DNA | | |
| | glycosylase I | | |
| MPG protein | Human 3- | 3-methyladenine, 7- | − |
| | methyladenine- | methylguanine, 3- | |
| | DNA | methylguanine, | |
| | glycosylase, | ethenoadenine, | |
| | ANPG protein, | ethenoguanine, | |
| | AAG protein, | hypoxanthine and | |
| | NMPG protein | chloroethylnitrosourea | |
| | | adducts | |
| UDG protein | E. coli Ung | uracil and 5-hydroxyuracil | + |
| | protein | | |
| Mut Y protein | E. coli MicA | adenine-guanine or adenine | + |
| | protein | cytosine mismatches | |
| T4 | PD-DNA | cis-syn cyclobutane | + |
| endonuclease | glycosylase | pyrimidine dimers | |
| V | | | |
| cv-PDG | | cis-syn and trans-syn-II | + |
| | | cyclobutane pyrimidine | |
| | | dimers | |

The substrates for the NER enzymes are a wide variety of bulky distortive DNA adducts and certain nondistortive types of DNA damage. The damage during NER is released as a part of an oligonucleotide fragment. Examples of nucleotide excision repair enzymes include the E. coli UvrABC exonuclease, which recognizes a wide spectrum of genotoxic DNA adducts. In addition to pyrimidine dimers and 6-4 photoproducts, the substrates of the Uvr ABC exonuclease include adducts of psoralen, 4-nitroquinoline oxide, cisplatin, benzo[a]pyrene diolepoxide (BPDE), aflatoxin B1, N-acetoxy-2-acetylaminofluorene, 7,12-dimethylbenzo[a]anthracene diolepoxide, mitomycin C, and many others. The Uvr ABC exonuclease complex consists of three proteins (UvrA, UvrB, and UvrC), which recognize and release the damage-containing fragment in a multi-step bimodal incision reaction. The excised oligonucleotide has a size of 12-13 nucleotides. However, in human cells, the damaged sequence is released within a 24-32 mer oligonucleotide.

The third major DNA repair mechanism, MMR, corrects single mispaired nucleotides and short loops. In addition to the excision repair systems, other important repair pathways, including direct reversal of DNA damage (O⁶-methylguanine-DNA methyltransferase and DNA photolyase) and double-strand break/recombination repair, are also fundamental factors in maintaining genetic stability.

A nuclease is generally present in an amount sufficient to cause the cleavage of the oligonucleotide, when it is reversibly hybridized to the polynucleotide analyte, to proceed at least half as rapidly as the maximum rate achievable with excess enzyme, preferably, at least 75% of the maximum rate. The concentration of the 5′-nuclease is usually determined empirically. Preferably, a concentration is used that is sufficient such that further increase in the concentration does not decrease the time for the amplification by over 5-fold, preferably 2-fold. The primary limiting factor generally is the cost of the reagent. In this respect, then, the polynucleotide analyte, or at least the target polynucleotide sequence, and the enzyme are generally present in a catalytic amount.

The probe that is cleaved by the enzyme is usually in large excess, preferably, 10⁻⁹ M to 10⁻⁵ M, and is used in an amount that maximizes the overall rate of its cleavage in accordance with the present invention wherein the rate is at least 10%, preferably, 50%, more preferably, 90%, of the maximum rate of reaction possible. Concentrations of the probe lower than 50% may be employed to facilitate detection of the fragments produced in accordance with the present invention. The amount of probe is at least as great as the number of molecules of product desired. Usually, the concentration of the probe is 0.1 nanomolar to 1 millimolar, preferably, 1 nanomolar to 10 micromolar. It should be noted that increasing the concentration of the probe causes the reaction rate to approach a limiting value that depends on the probe sequence, the temperature, the concentration of the target polynucleotide sequence and the enzyme concentration. For many detection methods very high concentrations of the probe may make detection more difficult.

In carrying out the methods in accordance with the present invention, an aqueous medium is employed. The pH for the medium is usually in the range of about 4.5 to 9.5, more usually in the range of about 5.5-8.5, and preferably in the range of about 6-8. The pH and temperature are chosen so as to achieve the reversible hybridization or equilibrium state under which cleavage of a probe occurs in accordance with the present invention. In some instances, a compromise is made in the reaction parameters in order to optimize the speed, efficiency, and specificity of these steps of the present method. Various buffers may be used to achieve the desired pH and maintain the pH during the determination. Illustrative buffers include borate, phosphate, carbonate, Tris, barbital and the like. The particular buffer employed is not critical to this invention but in individual methods one buffer may be preferred over another.

As mentioned above the reaction in accordance with the present invention is carried out under isothermal conditions. The reaction is generally carried out at a temperature that is near the melting temperature of the probe:polynucleotide complex. Accordingly, the temperature employed depends on a number of factors. Usually, for cleavage of the probe in accordance with the present invention, the temperature is about 35° C. to 90° C. depending on the length and sequence of the probe. It will usually be desired to use relatively high temperature of 60° C. to 85° C. to provide for a high rate of reaction. The amount of the fragments formed depends on the incubation time and temperature. In general, a moderate temperature is normally employed for carrying out the methods. The exact temperature utilized also varies depending on the salt concentration, pH, solvents used, and the length of and composition of the target polynucleotide sequence as well as the probe as mentioned above. It is understood that the selection of optimal reaction temperature takes into account the temperature dependence of the nuclease being employed.

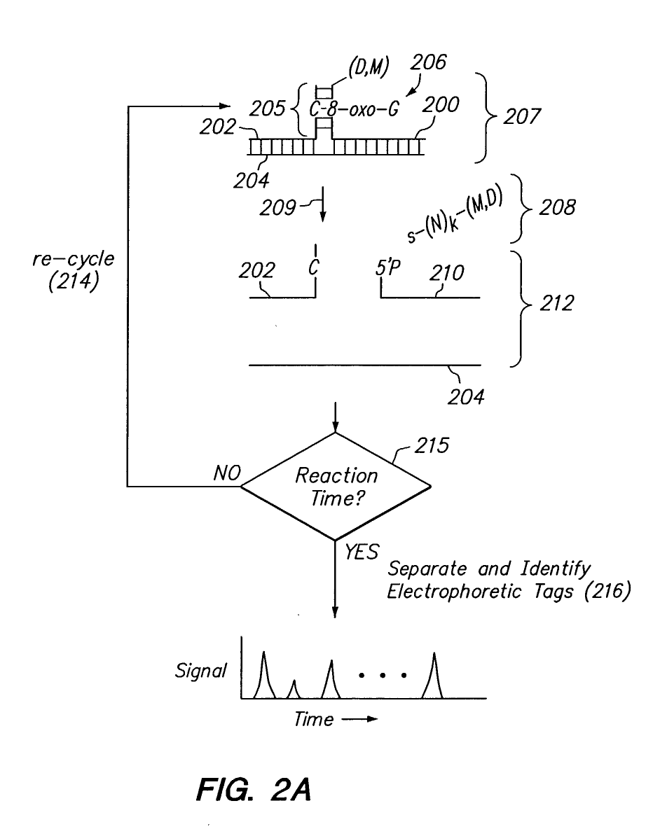

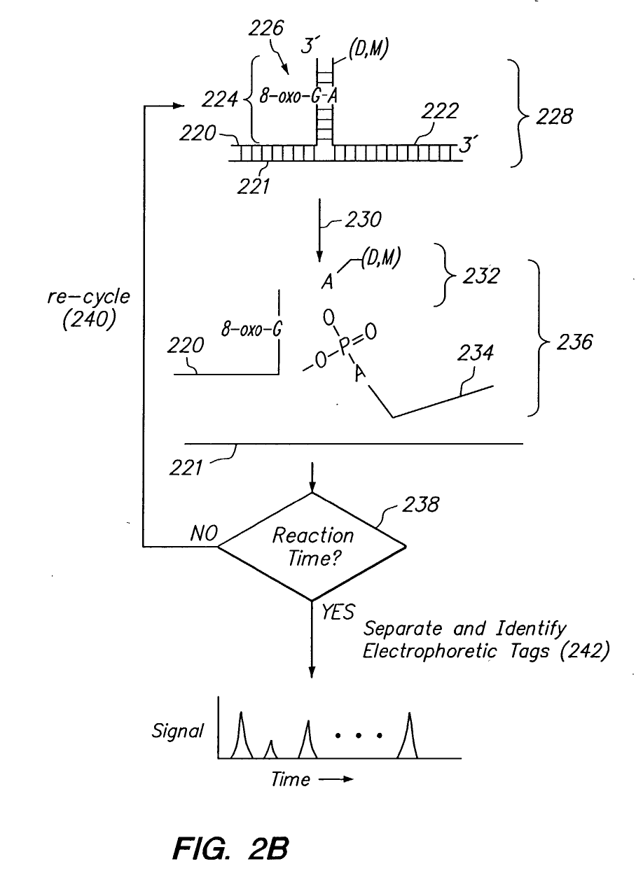

Particularly preferred protein nucleases from cleaving recognition duplexes include Fpg protein, Mut Y protein, hOGG1 protein, Nth protein (endonuclease III), human AP endonuclease, RNase H, and lambda endonuclease. Embodiments of the invention employing two of these nucleases are illustrated in FIGS. 2A-2B.

In FIG. 2A, an embodiment of the invention using hOGG1 protein as a cleavage agent is illustrated. Helper probe (202) and electrophoretic probe (200) are combined under assay conditions that permit the formation of a stable complex (207) with target polynucleotide (204). Preferably, electrophoretic probe (200) of the invention is defined by the formula:

3′-(N)ⱼ—Z—(N)ₖ—(M,D)

where N is a nucleotide, j is an integer in the range of from 8 to 40, k is an integer in the range of from 1 to 3; Z is a modified nucleoside recognized by hOGG1 protein when in a recognition duplex, preferably Z is 7,8-dihydro-8-oxo-2′-deoxyguanosine (“8-oxo-G”), foramidopyrimidine guanosine, or methylforamidopyrimidine guanosine; and (M,D) is described more fully below. Preferably, at least one nucleotide in the moiety “3′-(N)ⱼ” has a capture ligand attached to exclude uncleaved probe or non-tag fragments (210) from separation. Preferably, the capture ligand is biotin and the capture agent is streptavidin.

Complex (207) includes a recognition duplex (205) which includes a deoxycytosine:8-oxo-G basepair. Recognition duplex (205) is recognized by hOGG1 protein and 8-oxo-G is excised (209) releasing an electrophoretic tag (208) and cleavage fragment (210) having a 5′ phosphate. Preferably, electrophoretic tag (208) of the invention is defined by the formula:

3′-s-(N)ₖ—(M,D)

where “s” is an open ring sugar comprising five carbon atoms and two oxygen atoms, N is a nucleotide, k is an integer in the range of from 1 to 3, and (M,D) is a mobility modifying group and a detectable label that are described more fully below. Preferably, the structure “—(M,D)” is attached to (N)ₖ by a phosphate linker. Electrophoretic probes (200) of this embodiment may be synthesized using conventeional phosphoramidite chemistry as described below, where in particular 8-oxo-G phosphoramidite monomers are made as disclosed by Koizume et al, Nucleosides and Nucleotides, 13: 1517-1534 (1994); Kohda et al, Chem. Res. Toxicol., 9: 1278-1284 (1996); or the like. The cleavage or exchange of electrophoretic probe (200) causes the de-stabilization (212) of complex (207) so that target polynucleotide (204) becomes available to re-cycle (214) in another complex (207). Preferably, as taught by Western et al. U.S. Pat. No. 6,121,001, providing eletrophoretic probe (200) is high molar excess of the target or helper probe (202) enhances re-cycling (214). The reaction continues (215) for a time until a sufficient quantity of released electrophoretic tags are accumulated. The reaction time is determined empirically and depend of parameters that would be readily manipulated by one of ordinary skill in the art, such as reaction temperature, nuclease concentration, helper probe concentration, electrophoretic probe concentration, salt concentration, probe lengths and compositions, and the like. When the reaction is ended, electrophoretic tags are separated from the assay mixture and from one another for detection. As described more fully below, the separation step preferably includes a step for excluding material from the assay mixture that interferes with the separation or detection of the released electrophoretic tags. Such step includes (1) attaching a quencher to electrophoretic probes so that a fluorescent label of uncleaved probes is undetectable if it is separated with released electrophoretic tags, (2) attaching a capture ligand to electrophoretic probes, preferably on the probe opposite the site of cleavage, which capture ligand is combined with a reciprocal binding agent or receptor that imparts a charge to the bound probe or fragment that is opposite the charge of a released electrophoretic tag (for electrophoretic separation), (3) filtering larger molecular weight compounds or particulate matter to exclude it from being separated, and the like.

After the reaction is stopped, electrophoretic tags (208) are separated and identified (216), as described more fully below.

In FIG. 2A, an embodiment of the invention using MutY protein as a cleavage agent is illustrated. Helper probe (220) and electrophoretic probe (222) are combined under assay conditions that permit the formation of a stable complex (228) with target polynucleotide (221). Preferably, electrophoretic probe (222) of the invention is defined by the formula:

3′-(N)ⱼ—A—(N)ₖ—(M,D)

where N is a nucleotide, j is an integer in the range of from 8 to 40, k is an integer in the range of from 1 to 3 and (M,D) is described more fully below. As above, preferably, at least one nucleotide in the moiety “3′-(N)ⱼ” has a capture ligand attached to exclude uncleaved probe or non-tag fragments (234) from separation. Preferably, the capture ligand is biotin and the capture agent is streptavidin.

Helper probe (220) of the invention is defined by the formula:

5′-(N)ⱼ—Z′—(N)ₖ-3′

where N, k, and j are defined as above, and Z′ (226) is a modified nucleoside recognized by mut Y protein when base paired with deoxyadenosine in a recognition duplex, preferably Z′ is 7,8-dihydro-8-oxo-2′-deoxyguanosine (“8-oxo-G”).

Complex (228) includes a recognition duplex (224) which includes a deoxyadenosine:8-oxo-G basepair. Recognition duplex (224) is recognized by mut Y protein and the deoxyadenosine base paired with the 8-oxo-G is excised releasing electrophoretic tag (232) and cleavage fragment (234) having a 5′ phosphate. Preferably, electrophoretic tag (232) of the invention is defined by the formula:

3′-A—(N)ₖ—(M,D)

where A is deoxyadenosine, N is a nucleotide, k is an integer in the range of from 1 to 3, and (M,D) is a mobility modifying group and a detectable label that are described more fully below. Preferably, the structure “—(M,D)” is attached to (N)ₖ by a phosphate linker. Helper probe (220) of this embodiment may be synthesized using conventeional phosphoramidite chemistry as described above. The cleavage or exchange of electrophoretic probe (222) causes the de-stabilization (230) of complex (228) so that target polynucleotide (221) becomes available to re-cycle (240) in another complex (228). Again, as taught by Western et al. U.S. Pat. No. 6,121,001, providing eletrophoretic probe (222) in high molar excess of the target or helper probe (220) enhances re-cycling (240). The reaction continues (238) for a time until a sufficient quantity of released electrophoretic tags are accumulated. The reaction time is determined empirically and depends on parameters that are readily manipulated by one of ordinary skill in the art, such as reaction temperature, nuclease concentration, helper probe concentration, electrophoretic probe concentration, salt concentration, probe lengths and compositions, and the like. When the reaction is ended, electrophoretic tags are separated (242) from the assay mixture and from one another for detection. Optionally, as described above, additional steps may be taken to exclude interfering material from separation of the released electrophoretic tags.

Electrophoretic Probes and Tags

In accordance with the invention, a plurality of pairs of helper probes and electrophoretic probes are used to detect and/or measure the quantities of multiple target polynucleotides in a sample. The number of pairs of probes may be the same or larger than the number of target polynucleotides sought to be detected. In particular, more than one pair of probes may be directed to the same target polynucleotide. The number of pairs of probes in an assay may range from 2 to 100, preferably from 5 to 50, and more preferably from 10 to 30. Generally, electrophoretic probes of the invention are oligonucleotides having various modifications including the attachment of one or more reporter groups that when cleaved become electrophoretic tags that are separated and identified.

Electrophoretic tag, E, is a water soluble organic compound that is stable with respect to the active species, especially singlet oxygen, and that includes a detection or reporter group. Otherwise, E may vary widely in size and structure. Preferably, E carries a charge at neutral pH and has a molecular weight in the range of from about 150 to about 10,000 daltons, more preferably, from about 150 to about 5000 daltons, and most preferably, from about 150 to 2500 daltons. Preferred structures of E are described more fully below. Preferably, the detection group generates an electrochemical, fluorescent, or chromogenic signal. Most preferably, the detection group generates a fluorescent signal. Compositions of the invention include pluralities of electrophoretic tags that may be used together to carry out the multiplexed assays of the invention. Preferably, the plurality of electrophoretic tags in a composition is at least 5, and more preferably, at least 10. Still more preferably, the plurality is in the range of from 5 to 200, and more preferably, from 5 to 100, or 5 to 75, or from 5 to 50, or from 10 to 30. Preferably, electrophoretic tags within a plurality of a composition each have either a unique charge-to-mass ratio and/or a unique optical property with respect to the other members of the same plurality. Preferably, the optical property is a fluorescence property, such as emission spectrum, fluorescence lifetime, or the like. More preferably, the fluorescence property is emission spectrum. For example, each electrophoretic tag of a plurality may have the same fluorescent emission properties, but each will differ from one another by virtue of unique charge-to-mass ratios. On the other hand, or two or more of the electrophoretic tags of a plurality may have identical charge-to-mass ratios, but they will have unique fluorescent properties, e.g. spectrally resolvable emission spectra, so that all the members of the plurality are distinguishable by the combination of electrophoretic separation and fluorescence measurement.

Preferably, electrophoretic tags in a plurality are detected by electrophoretic separation and fluorescence. Preferably, electrophoretic tags having substantially identical fluorescence properties have different electrophoretic mobilities so that distinct peaks in an electropherogram are formed under separation conditions. A measure of the distinctness, or lack of overlap, of adjacent peaks is electrophoretic resolution, which is the distance between adjacent peak maximums divided by four times the larger of the two standard deviations of the peaks. Preferably, adjacent peaks have a resolution of at least 1.0, and more preferably, at least 1.5, and most preferably, at least 2.0. In a given separation and detection system, the desired resolution may be obtained by selecting a plurality of electrophoretic tags whose members have electrophoretic mobilities that differ by at least a peak-resolving amount, such quantity depending on several factors well known to those of ordinary skill, including signal detection system, nature of the fluorescent moieties, the diffusion coefficients of the tags, the presence or absence of sieving matrices, nature of the electrophoretic apparatus, e.g. presence or absence of channels, length of separation channels, and the like. Preferably, pluralities of electrophoretic tags of the invention are separated by conventional capillary electrophoresis apparatus, either in the presence or absence of a conventional sieving matix. Exemplary capillary electroresis apparatus include Applied Biosystems (Foster City, Calif.) models 310, 3100 and 3700; Beckman (Fullerton, Calif.) model P/ACE MDQ; Amersham Biosciences (Sunnyvale, Calif.) MegaBACE 1000 or 4000; SpectruMedix genetic analysis system; and the like. Preferably, in such conventional apparatus, the electrophoretic mobilities of electrophoretic tags of a plurality differ by at least one percent, and more preferably, by at least a percentage in the range of from 1 to 10 percent.

Electrophoretic mobility is proportional to q/M^{2/3}, where q is the charge on the molecule and M is the mass of the molecule. Desirably, the difference in mobility under the conditions of the determination between the closest electrophoretic labels will be at least about 0.001, usually 0.002, more usually at least about 0.01, and may be 0.02 or more.

A preferred structure of electrophoretic tag, E, is (M, D), where M is a mobility-modifying moiety and D is a detection moiety. The notation “(M, D)” is used to indicate that the ordering of the M and D moieties may be such that either moiety can be adjacent to the cleavable linkage, L. That is, “T-L-(M, D)” designates electrophoretic probe of either of two forms: “T-L-M-D” or “T-L-D-M.”

Detection moiety, D, may be a fluorescent label or dye, a chromogenic label or dye, an electrochemical label, or the like. Preferably, D is a fluorescent dye. Exemplary fluorescent dyes for use with the invention include water-soluble rhodamine dyes, fluoresceins, 4,7-dichlorofluoresceins, benzoxanthene dyes, and energy transfer dyes, disclosed in the following references: Handbook of Molecular Probes and Research Reagents, 8th ed., (Molecular Probes, Eugene, 2002); Lee et al, U.S. Pat. No. 6,191,278; Lee et al, U.S. Pat. No. 6,372,907; Menchen et al, U.S. Pat. No. 6,096,723; Lee et al, U.S. Pat. No. 5,945,526; Lee et al, Nucleic Acids Research, 25: 2816-2822 (1997); Hobb, Jr., U.S. Pat. No. 4,997,928; Khanna et al., U.S. Pat. No. 4,318,846; Reynolds, U.S. Pat. No. 3,932,415; Eckert et al, U.S. Pat. No. 2,153,059; Eckert et al, U.S. Pat. No. 2,242,572; Taing et al, International patent publication WO 02/30944; and the like. Further specific exemplary fluorescent dyes include 5- and 6-carboxyrhodamine 6G; 5- and 6-carboxy-X-rhodamine, 5- and 6-carboxytetramethylrhodamine, 5- and 6-carboxyfluorescein, 5- and 6-carboxy-4,7-dichlorofluorescein, 2′,7′-dimethoxy-5- and 6-carboxy-4,7-dichlorofluorescein, 2′,7′-dimethoxy-4′,5′-dichloro-5- and 6-carboxyfluorescein, 2′,7′-dimethoxy-4′,5′-dichloro-5- and 6-carboxy-4,7-dichlorofluorescein, 1′,2′,7′,8′-dibenzo-5- and 6-carboxy-4,7-dichlorofluorescein, 1′,2′,7′,8′-dibenzo-4′,5′-dichloro-5- and 6-carboxy-4,7-dichlorofluorescein, 2′,7′-dichloro-5- and 6-carboxy-4,7-dichlorofluorescein, and 2′,4′,5′,7′-tetrachloro-5- and 6-carboxy-4,7-dichlorofluorescein. Most preferably, D is a fluorescein or a fluorescein derivative.

M is generally a chemical group or moiety that has or is designed to have a particular charge-to-mass ratio, and thus a particular electrophoretic mobility in a defined electrophoretic system. Exemplary types of mobility-modifying moieties are discussed below. In a set of n electrophoretic probes, each unique mobility modifier is designated Mⱼ, where j=1 to n, and n has a value as described above. The mobility-modifying moiety may be considered to include a mass-modifying region and/or a charge-modifying region or a single region that acts as both a mass- and charge-modifying region. In the probe sets utilized in the invention, the mobility-modifying moiety may have one or more of the following characteristics: (i) a unique charge-to-mass ratio due to variations in mass, but not charge; (ii) a unique charge-to-mass ratio due to changes in both mass and charge; and (iii) a unique charge-to-mass ratios of between about −0.0001 and about 0.5, usually, about −0.001 and about 0.1. As noted above, D is typically common among a set or plurality of different electrophoretic probes, but may also differ among probe sets, contributing to the unique electrophoretic mobilities of the released electrophoretic tag.

The size and composition of mobility-modifying moiety, M, can vary from a bond to about 100 atoms in a chain, usually not more than about 60 atoms, more usually not more than about 30 atoms, where the atoms are carbon, oxygen, nitrogen, phosphorous, boron and sulfur. Generally, when other than a bond, the mobility-modifying moiety has from about 0 to about 40, more usually from about 0 to about 30 heteroatoms, which in addition to the heteroatoms indicated above may include halogen or other heteroatom. The total number of atoms other than hydrogen is generally fewer than about 200 atoms, usually fewer than about 100 atoms. Where acid groups are present, depending upon the pH of the medium in which the mobility-modifying moiety is present, various cations may be associated with the acid group. The acids may be organic or inorganic, including carboxyl, thionocarboxyl, thiocarboxyl, hydroxamic, phosphate, phosphite, phosphonate, phosphinate, sulfonate, sulfinate, boronic, nitric, nitrous, etc. For positive charges, substituents include amino (includes ammonium), phosphonium, sulfonium, oxonium, etc., where substituents are generally aliphatic of from about 1-6 carbon atoms, the total number of carbon atoms per heteroatom, usually be less than about 12, usually less than about 9. The side chains include amines, ammonium salts, hydroxyl groups, including phenolic groups, carboxyl groups, esters, amides, phosphates, heterocycles. M may be a homo-oligomer or a hetero-oligomer, having different monomers of the same or different chemical characteristics, e.g., nucleotides and amino acids.

The charged mobility-modifying moieties generally have only negative or positive charges, although one may have a combination of charges, particularly where a region to which the mobility-modifying moiety is attached is charged and the mobility-modifying moiety has the opposite charge. The mobility-modifying moieties may have a single monomer that provides the different functionalities for oligomerization and carry a charge or two monomers may be employed, generally two monomers. One may use substituted diols, where the substituents are charged and dibasic acids. Illustrative of such oligomers is the combination of diols or diamino, such as 2,3-dihydroxypropionic acid, 2,3-dihydroxysuccinic acid, 2,3-diaminosuccinic acid, 2,4-dihydroxyglutaric acid, etc. The diols or diamino compounds can be linked by dibasic acids, which dibasic acids include the inorganic dibasic acids indicated above, as well as dibasic acids, such as oxalic acid, malonic acid, succinic acid, maleic acid, furmaric acid, carbonic acid, etc. Instead of using esters, one may use amides, where amino acids or diamines and diacids may be employed. Alternatively, one may link the hydroxyls or amines with alkylene or arylene groups.

By employing monomers that have substituents that provide for charges, or which may be modified to provide charges, one can provide for mobility-modifying moieties having the desired charge-to-mass ratio. For example, by using serine or threonine, one may modify the hydroxyl groups with phosphate to provide negatively charged mobility-modifying moieties. With arginine, lysine and histidine, one provides for positively charged mobility-modifying moieties. Oligomerization may be performed in conventional ways to provide the appropriately sized mobility-modifying moiety. The different mobility-modifying moieties having different orders of oligomers, generally having from 1 to 20 monomeric units, more usually about 1 to 12, where a unit intends a repetitive unit that may have from 1 to 2 different monomers. For the most part, oligomers may be used with other than nucleic acid target-binding regions. The polyfunctionality of the monomeric units provides for functionalities at the termini that may be used for conjugation to other moieties, so that one may use the available functionality for reaction to provide a different functionality. For example, one may react a carboxyl group with an aminoethylthiol, to replace the carboxyl group with a thiol functionality for reaction with an activated olefin.

By using monomers that have about 1 to about 3 charges, one may employ a low number of monomers and provide for mobility variation with changes in molecular weight. Of particular interest are polyolpolycarboxylic acids having from about two to four of each functionality, such as tartaric acid, 2,3-dihydroxyterephthalic acid, 3,4-dihydroxyphthalic acid, Δ⁵-tetrahydro-3,4-dihydroxyphthalic acid, etc. To provide for an additional negative charge, these monomers may be oligomerized with a dibasic acid, such as a phosphoric acid derivative to form the phosphate diester. Alternatively, the carboxylic acids could be used with a diamine to form a polyamide, while the hydroxyl groups could be used to form esters, such as phosphate esters, or ethers such as the ether of glycolic acid, etc. To vary the mobility, various aliphatic groups of differing molecular weight may be employed, such as polymethylenes, polyoxyalkylenes, polyhaloaliphatic or aromatic groups, polyols, e.g., sugars, where the mobility will differ by at least about 0.01, more usually at least about 0.02 and more usually at least about 0.5.

In another aspect, (M,D) moieties are constructed from chemical scaffolds used in the generation of combinatorial libraries. For example, the following references describe scaffold compound useful in generating diverse mobility modifying moieties: peptoids (PCT Publication No WO 91/19735, Dec. 26, 1991), encoded peptides (PCT Publication WO 93/20242, Oct. 14 1993), random bio-oligomers (PCT Publication WO 92/00091, Jan. 9, 1992), benzodiazepines (U.S. Pat. No. 5,288,514), diversomeres such as hydantoins, benzodiazepines and dipeptides (Hobbs DeWitt, S. et al., Proc. Nat. Acad. Sci. U.S.A. 90: 6909-6913 (1993), vinylogous polypeptides (Hagihara et al. J.Amer. Chem. Soc. 114: 6568 (1992)), nonpeptidal peptidomimetics with a Beta-D-Glucose scaffolding (Hirschmann, R. et al., J.Amer. Chem. Soc. 114: 9217-9218 (1992)), analogous organic syntheses of small compound libraries (Chen, C. et al. J.Amer. Chem. Soc. 116: 2661(1994)), oligocarbamates (Cho, C. Y. et al. Science 261: 1303(1993)), peptidyl phosphonates (Campbell, D. A. et al., J. Org. Chem. 59:658(1994)); Cheng et al, U.S. Pat. No. 6,245,937; Heizmann et al, “Xanthines as a scaffold for molecular diversity,” Mol. Divers. 2: 171-174 (1997); Pavia et al, Bioorg. Med. Chem., 4: 659-666 (1996); Ostresh et al, U.S. Pat. No. 5,856,107; Gordon, E. M. et al., J. Med. Chem. 37: 1385 (1994); and the like. Preferably, in this aspect, D is a substituent on a scaffold and M is the rest of the scaffold.

In yet another aspect, (M, D) moieties are constructed from one or more of the same or different common or commercially available linking, cross-linking, and labeling reagents that permit facile assembly, especially using a commercial DNA or peptide synthesizer for all or part of the synthesis. In this aspect, (M, D) moieties are made up of subunits usually connected by phosphodiester and amide bonds. Exemplary, precusors include, but are not limited to, dimethoxytrityl (DMT)-protected hexaethylene glycol phosphoramidite, 6-(4-Monomethoxytritylamino)hexyl-(2-cyanoethyl)-(N,N-diisopropyl)-phosphoramidite, 12-(4-Monomethoxytritylamino)dodecyl-(2-cyanoethyl)-(N,N-diisopropyl)-phosphoramidite, 2-[2-(4-Monomethoxytrityl)aminoethoxy]ethyl-(2-cyanoethyl), N,N-diisopropyl)-phosphoramidite, (S-Trityl-6-mercaptohexyl)-(2-cyanoethyl)-(N,N-diisopropyl)-phosphoramidite, 5′-Fluorescein phosphoramidite, 5′-Hexachloro-Fluorescein Phosphoramidite, 5′-Tetrachloro-Fluorescein Phosphoramidite, 9-O-Dimethoxytrityl-triethylene glycol,1-[(2-cyanoethyl)-(N,N-diisopropyl)]-phosphoramidite, 3(4,4′Dimethoxytrityloxy)propyl-1-[(2-cyanoethyl)-(N,N-diisopropyl)]-phosphoramidite, 5′-O-Dimethoxytrityl-1′,2′-Dideoxyribose-3′-[(2-cyanoethyl)-(N,N-diisopropyl)]-phosphoramidite, 18-O Dimethoxytritylhexaethyleneglycol,1-[(2-cyanoethyl)-(N,N-diisopropyl)]-phosphoramidite, 12-(4,4′-Dimethoxytrityloxy)dodecyl-1-[(2-cyanoethyl)-(N,N-diisopropyl)]-phosphoramidite, 1,3-bis-[5-(4,4′-dimethoxytrityloxy)pentylamido]propyl-2-[(2-cyanoethyl)-(N,N-diisopropyl)]-phosphoramidite, 1-[5-(4,4′-dimethoxytrityloxy)pentylamido]-3-[5-fluorenomethoxycarbonyloxy pentylamido]-propyl-2-[(2-cyanoethyl)-(N,N-diisopropyl)]-phosphoramidite, Tris-2,2,2-[3-(4,4′-dimethoxytrityloxy)propyloxymethyl]ethyl-[(2-cyanoethyl)-(N,N-diisopropyl)]-phosphoramidite, succinimidyl trans-4-(maleimidylmethyl) cyclohexane-1-carboxylate (SMCC), succinimidyl 3-(2-pyridyldithio)propionate (SPDP), succinimidyl acetylthioacetate, Texas Red-X-succinimidyl ester, 5- and 6-carboxytetramethylrhodamine succinimidyl ester, bis-(4-carboxypiperidinyl)sulfonerhodamine di(succinimidyl ester), 5- and 6-((N-(5-aminopentyl)aminocarbonyl)tetramethylrhodamine, succinimidyl 4-(p-maleimidophenyl)butyrate (SMPB); N-γ-maleimidobutyryl-oxysuccinimide ester (GMBS); p-nitrophenyl iodoacetate (NPIA); 4-(4-N-maleimidophenyl)butyric acid hydrazide (MPBH); and like reagents. The above reagents are commercially available, e.g. from Glen Research (Sterling, Va.), Molecular Probes (Eugene, Oreg.), Pierce Chemical, and like reagent providers. Use of the above reagents in conventional synthetic schemes is well known in the art, e.g. Hermanson, Bioconjugate Techniques (Academic Press, New York, 1996). In particular, M may be constructed from the following reagents: dimethoxytrityl (DMT)-protected hexaethylene glycol phosphoramidite, 6-(4-Monomethoxytritylamino)hexyl-(2-cyanoethyl)-(N,N-diisopropyl)-phosphoramidite, 12-(4-Monomethoxytritylamino)dodecyl-(2-cyanoethyl)-(N,N-diisopropyl)-phosphoramidite, 2-[2-(4-Monomethoxytrityl)aminoethoxy]ethyl-(2-cyanoethyl), N,N-diisopropyl)-phosphoramidite, (S-Trityl-6-mercaptohexyl)-(2-cyanoethyl)-(N,N-diisopropyl)-phosphoramidite, 9-O-Dimethoxytrityl-triethylene glycol,1-[(2-cyanoethyl)-(N,N-diisopropyl)]-phosphoramidite, 3(4,4Dimethoxytrityloxy)propyl-1-[(2-cyanoethyl)-(N,N-diisopropyl)]-phosphoramidite, 5′-O-Dimethoxytrityl-1′,2′-Dideoxyribose-3′-[(2-cyanoethyl)-(N,N-diisopropyl)]-phosphoramidite, 18-O Dimethoxytritylhexaethyleneglycol,1-[(2-cyanoethyl)-(N,N-diisopropyl)]-phosphoramidite, 12-(4,4′-Dimethoxytrityloxy)dodecyl-1-[(2-cyanoethyl)-(N,N-diisopropyl)]-phosphoramidite, 1,3-bis-[5-(4,4′-dimethoxytrityloxy)pentylamido]propyl-2-[(2-cyanoethyl)-(N,N-diisopropyl)]-phosphoramidite, 1-[5-(4,4′-dimethoxytrityloxy)pentylamido]-3-[5-fluorenomethoxycarbonyloxy pentylamido]-propyl-2-[(2-cyanoethyl)-(N,N-diisopropyl)]-phosphoramidite, Tris-2,2,2-[3-(4,4′-dimethoxytrityloxy)propyloxymethyl]ethyl-[(2-cyanoethyl)-(N,N-diisopropyl)]-phosphoramidite, succinimidyl trans-4-(maleimidylmethyl) cyclohexane-1-carboxylate (SMCC), succinimidyl 3-(2-pyridyldithio)propionate (SPDP), succinimidyl acetylthioacetate, succinimidyl 4-(p-maleimidophenyl)butyrate (SMPB); N-γ-maleimidobutyryl-oxysuccinimide ester (GMBS); p-nitrophenyl iodoacetate (NPIA); and 4-(4-N-maleimidophenyl)butyric acid hydrazide (MPBH).

M may also comprise polymer chains prepared by known polymer subunit synthesis methods. Methods of forming selected-length polyethylene oxide-containing chains are well known, e.g. Grossman et al, U.S. Pat. No. 5,777,096. It can be appreciated that these methods, which involve coupling of defined-size, multi-subunit polymer units to one another, directly or via linking groups, are applicable to a wide variety of polymers, such as polyethers (e.g., polyethylene oxide and polypropylene oxide), polyesters (e.g., polyglycolic acid, polylactic acid), polypeptides, oligosaccharides, polyurethanes, polyamides, polysulfonamides, polysulfoxides, polyphosphonates, and block copolymers thereof, including polymers composed of units of multiple subunits linked by charged or uncharged linking groups. In addition to homopolymers, the polymer chains used in accordance with the invention include selected-length copolymers, e.g., copolymers of polyethylene oxide units alternating with polypropylene units. As another example, polypeptides of selected lengths and amino acid composition (i.e., containing naturally occurring or man-made amino acid residues), as homopolymers or mixed polymers.

In another aspect, the detection moiety of (M,D) generates a fluorescent signal by an energy transfer mechanism. Preferably, in this aspect, D has the form “D₁-g-D₂” where D₁ and D₂ are acceptor-donor pairs of molecules, e.g. Wu et al, Anal. Biochem., 218: 1-13 (1994), and g is a rigid linker that maintains D₁ and D₂ at a substantially constant distance. Guidance in selecting rigid linker, g, may be found in We et al (cited above) and in U.S. Pat. Nos. 5,863,727; 5,800,996; 5,945,526; and 6,008,379. Either D₁ or D₂ may be the acceptor and the other the donor molecule in the pair. Exemplary, energy transfer detection moieties for use with the invention are disclosed in Lee et al, U.S. Pat. No. 5,945,526; Lee et al, Nucleic Acids Research, 25: 2816-2822 (1997); Taing et al, International patent publication WO 02/30944; and like references. Preferably, rigid linker, g, is selected so that the distance between D₁ and D₂ is maintained at a substantially constant distance within the range of from 10-100 Angstroms. A wide variety of linking groups may be employed with the proviso that the linkage be stable to the presence of singlet oxygen. Preferably, D₁ and D₂ are selected from the set of fluorescein, rhodamine, rhodamine 6G, rhodamine 110, rhodamine X, tetramethylrhodamine, and halogenated derivatives thereof. More preferably, D₁ and D₂ are both fluorescein dyes.

In one aspect, g may be selected from any of R₁—R₂—R₁ and R₁—R₂—C(═O)—X₁—R₃, the latter being present in either orientation with respect to D₁ and D₂; where X₁ is O, S, or NH; R₁ is (C₁-C₅ alkyldiyl, X₁, C(═O)) such that any one to three the moieties in parentheses are arranged in any linear order; R₂ is a 5 to 6 membered ring selected from the group consisting of cyclopentene, cyclohexene, cyclopentadiene, cyclohexadiene, furan, pyrrole, isopyrole, isoazole, pyrazole, isoimidazole, pyran, pyrone, benzene, pyridine, pyridazine, pyrimidine, pyrazine oxazine, indene, benzofuran, thionaphthene, indole and naphthalene; R₃ is C₁-C₅ alkyldiyl.

Pluralities of electrophoretic tags may include oligopeptides for providing the charge, particularly oligopeptides of from 2-6, usually 2-4 monomers, either positive charges resulting from lysine, arginine and histidine or negative charges, resulting from aspartic and glutamic acid. Of course, one need not use naturally occurring amino acids, but unnatural or synthetic amino acids, such as taurine, phosphate substituted serine or threonine, S-α-succinylcysteine, co-oligomers of diamines and amino acids, etc.

In one embodiment of the present invention, the charge-imparting moiety is conveniently composed primarily of amino acids but also may include thioacids and other carboxylic acids having from one to five carbon atoms. The charge imparting moiety may have from about 1 to about 30, preferably about 1 to about 20, more preferably, about 1 to about 10 amino acids per moiety and may also comprise about 1 to about 3 thioacids or other carboxylic acids. However, when used with an uncharged sub-region, the charged sub-region will generally have from about 1 to about 4, frequently about 1 to about 3 amino acids. As mentioned above, any amino acid, both naturally occurring and synthetic, may be employed.

In a particular embodiment, T-L-M-D may be represented by the formula:

T—L-(amino acid)ₙ-L′-Fluorescer

wherein L′ is a bond or a linking group of from 1 to 20 atoms other than hydrogen, n is 1 to 20, and L is a cleavable linkage to the polypeptide-binding moiety. In this embodiment T is linked to the terminal amino acid by a cleavable linkage. An example of this embodiment, by way of illustration and not limitation, is one in which the fluorescer is fluorescein, L′ is a bond in the form of an amide linkage involving the meta-carboxyl of the fluorescein and the terminal amine group of lysine, and T is a polypeptide-binding moiety.

Examples of electrophoretic tags based on such label conjugates may be represented as follows:

Fluorescein-(CO)NH—(CH₂)₄—NH-(amino acid)ₙ

where formulas and charges at neutral pH for specific compounds are set forth in Table 2.

TABLE 2

| | | | Mol. Wt. | |
|---|---|---|---|---|
| No. | (amino acid)n | Charge (q) | (M) | q/M^{2/3} |
| 1 | none | −1 | 446 | −.0178 |
| 2 | lysine | −1 | 591 | −.0148 |
| 3 | (lysine)₂ | neutral | 737 | .0128 |
| 4 | alanine | −2 | 534 | −.0298 |
| 5 | aspartic acid | −3 | 578 | −.0423 |
| 6 | (aspartic acid)₂ | −4 | 711 | −.0491 |
| 7 | (aspartic acid)₃ | −5 | 844 | −.0877 |
| 8 | (aspartic acid)₄ | −6 | 977 | −.0595 |
| 9 | (aspartic acid)₅ | −7 | 1110 | −.0638 |
| 10 | (aspartic acid)₆ | −8 | 1243 | −.0675 |
| 11 | (aspartic acid)₇ | −9 | 1376 | −.0710 |
| 12 | alanine-lysine | −2 | 680 | −.0253 |
| 13 | aspartic acid- | −2 | 724 | −.0243 |
| | lysine | | | |
| 14 | (aspartic acid)₂- | −3 | 857 | −.0325 |
| | lysine | | | |
| 15 | (aspartic acid)₃- | −4 | 990 | −.0393 |
| | lysine | | | |
| 16 | (aspartic acid)₄- | −5 | 1123 | −.0452 |
| | lysine | | | |
| 17 | (aspartic acid)₅- | −6 | 1256 | −.0503 |
| | lysine | | | |
| 18 | (aspartic acid)₆- | −7 | 1389 | −.0549 |
| | lysine | | | |
| 19 | (aspartic acid)₇- | −8 | 1522 | −.0590 |
| | lysine | | | |
| 20 | (aspartic acid)₈- | −9 | 1655 | −.0627 |
| | lysine | | | |
| 21 | (lysine)₄ | +2 | 1029 | .0192 |
| 22 | (lysine)₅ | +3 | 1170 | .0264 |

wherein q is charge, M is mass and mobility is proportional to q/M^{2/3}.

In another embodiment, mobility-modifying moiety, M, is dependent on using an alkylene or aralkylene (comprising a divalent aliphatic group having about 1 to about 2 aliphatic regions and about 1 to about 2 aromatic regions, generally benzene), where the groups may be substituted or unsubstituted, usually unsubstituted, of from about 2 to about 16, more usually about 2 to about 12, carbon atoms, where the mobility-modifying moiety may link the same or different fluorescers to a monomeric unit, e.g., a nucleotide. The mobility-modifying moiety may terminate in a carboxy, hydroxy or amino group, being present as an ester or amide. By varying the substituents on the fluorophore, one can vary the mass in units of at least about 5 or more, usually at least about 9, so as to be able to obtain satisfactory separation in capillary electrophoresis. To provide further variation, a thiosuccinimide group may be employed to join alkylene or aralkylene groups at the nitrogen and sulfur, so that the total number of carbon atoms may be in the range of about 2 to about 30, more usually about 2 to about 20. Instead of or in combination with the above groups and to add hydrophilicity, one may use alkyleneoxy groups.

Besides the nature of the mobility-modifying moiety, as already indicated, diversity can be achieved by the chemical and optical characteristics of the label, the use of energy transfer complexes, variation in the chemical nature of the mobility-modifying moiety, which affects mobility, such as folding, interaction with the solvent and ions in the solvent, and the like. In one embodiment of the invention, the mobility-modifying moiety may be an oligomer, where the mobility-modifying moiety may be synthesized on a support or produced by cloning or expression in an appropriate host. Conveniently, polypeptides can be produced where there is only one cysteine or serine/threonine/tyrosine, aspartic/glutamic acid, or lysine/arginine/histidine, other than an end group, so that there is a unique functionality, which may be differentially functionalized. By using protective groups, one can distinguish a side-chain functionality from a terminal amino acid functionality. Also, by appropriate design, one may provide for preferential reaction between the same functionalities present at different sites on the mobility-modifying moiety. Whether one uses synthesis or cloning for preparation of oligopeptides, is to a substantial degree depend on the length of the mobility-modifying moiety.

Substituted aryl groups can serve as both mass- and charge-modifying regions. Various functionalities may be substituted onto the aromatic group, e.g., phenyl, to provide mass as well as charges to the electrophoretic tag. The aryl group may be a terminal group, where only one linking functionality is required, so that a free hydroxyl group may be acylated, may be attached as a side chain to an hydroxyl present on the electrophoretic tag, or may have two functionalities, e.g., phenolic hydroxyls, that may serve for phosphite ester formation and other substituents, such as halo, haloalkyl, nitro, cyano, alkoxycarbonyl, alkylthio, etc. where the groups may be charged or uncharged.

The labeled conjugates may be prepared utilizing conjugating techniques that are well known in the art. M may be synthesized from smaller molecules that have functional groups that provide for linking of the molecules to one another, usually in a linear chain. Such functional groups include carboxylic acids, amines, and hydroxy- or thiol-groups. In accordance with the present invention the charge-imparting moiety may have one or more side groups pending from the core chain. The side groups have a functionality to provide for linking to a label or to another molecule of the charge-imparting moiety. Common functionalities resulting from the reaction of the functional groups employed are exemplified by forming a covalent bond between the molecules to be conjugated. Such functionalities are disulfide, amide, thioamide, dithiol, ether, urea, thiourea, guanidine, azo, thioether, carboxylate and esters and amides containing sulfur and phosphorus such as, e.g., sulfonate, phosphate esters, sulfonamides, thioesters, etc., and the like.

The linkages of the components of the electrophoretic tag are discussed above. The linkage between the detectable moiety and the mobility-modifying moiety is generally stable to the action of the cleavage-inducing moiety, so that the mobility-modifying moiety and detectable moiety may be released as an intact unit from the electrophoretic probe during the cleavage of the electrophoretic tag from the electrophoretic probe.

For the most part, the mobility-modifying moiety may be a bond, where the detectable moiety or label is directly bonded to the target-binding moiety, or a link of from about 1 to about 500 or more, usually about 1 to about 300 atoms, more usually about 2 to about 100 atoms in the chain. In this embodiment, the total number of atoms in the chain will depend to a substantial degree on the diversity required to recognize all the targets to be determined. The chain of the mobility-modifying moiety for the most part is comprised of carbon, nitrogen, oxygen, phosphorous, boron, and sulfur. Various substituents may be present on the mobility-modifying moiety, which may be naturally present as part of the naturally occurring monomer or introduced by synthesis. Functionalities which may be present in the chain include amides, phosphate esters, ethers, esters, thioethers, disulfides, borate esters, sulfate esters, etc. The side chains include amines, ammonium salts, hydroxyl groups, including phenolic groups, carboxyl groups, esters, amides, phosphates, heterocycles, particularly nitrogen heterocycles, such as the nucleoside bases and the amino acid side chains, such as imidazole and quinoline, thioethers, thiols, or other groups of interest to change the mobility of the electrophoretic tag.

The mobility-modifying moiety may be a homo-oligomer or a hetero-oligomer compound having different monomers of the same or different chemical characteristics, e.g., nucleotides and amino acids. In one embodiment, the electrophoretic tags will have a linker, which provides the linkage between the mobility-modifying moiety and the detectable label molecule, usually a fluorescer, or a functionality that may be used for linking to a detectable label molecule. By having different functionalities, which may be individually bonded to a detectable label molecule, one enhances the opportunity for diversity of the electrophoretic tags. Using different fluorescers for joining to the different functionalities, the different fluorescers can provide differences in light emission and charge-to-mass ratios for the electrophoretic tags.

Capture Ligands

Capture ligands attached to electrophoretic probes may be used to impart a charge to uncleaved or partially cleaved probes that is different than, preferably opposite of, the charge of released electrophoretic tags. Such ligands may also be used to bind or adsorb uncleaved or partially cleaved probe, or other reaction components, to exclude such materials from being separated along with released electrophoretic tags.

Ligands and receptors include biotin and streptavidin, ligand and antiligand, e.g. digoxin or derivative thereof and antidigoxin, etc. By having a ligand conjugated to the oligonucleotide, one can sequester the electrophoretic probe/target complex with the receptor to the ligand, remove unhybridized probe and then release the bound electrophoretic tags. Alternatively, a receptor for the ligand that has a positive charge can be added to reaction products, wherein binding to the undegraded probe/target complex causes migration of the complex in the opposite direction of the released electrophoretic tags. Thus, an aspect of the present invention is providing electrophoretic probe that have a charge opposite that of a released electrophoretic tag. This is conveniently accomplished by providing a positively charged receptor or capture agent that binds to a capture ligand on uncleaved electrophoretic probes.

Conveniently, one will usually have a ligand of under about 1 kDa. This may be exemplified by the use of biotin as the ligand and avidin, which is highly positively charged, as the receptor (capture agent)/positively charged molecule. Instead of biotin/avidin, one may have other pairs, where the receptor, e.g. antibody, is naturally positively charged or is made so by conjugation with one or more positively charged entities, such as arginine, lysine or histidine, ammonium, etc. The presence of the positively charged moiety has many advantages in substantially removing the electrophoretic probes, comprising both undegraded and degraded probe.

If desired, the receptor may be used to physically sequester the molecules to which it binds, removing entirely intact electrophoretic probes containing the target-binding moiety or modified target-binding moieties retaining the ligand. These modified target-binding moieties may be as a result of degradation of the starting material, contaminants during the preparation, aberrant cleavage, etc. or other nonspecific degradation products of the target-binding sequence. As above, a ligand, exemplified by biotin, is attached to the target-binding moiety, e.g. the penultimate nucleoside, so as to be separated from the electrophoretic tag upon cleavage.

Other receptors include natural or synthetic receptors, such as immunoglobulins, lectins, enzymes, etc. Desirably, the receptor is positively charged, naturally as in the case of avidin, or is made so, by the addition of a positively charged moiety or moieties, such as ammonium groups, basic amino acids, etc. Avidin binds to the biotin attached to the detection probe and its degradation products. Avidin is positively charged, while the cleaved electrophoretic tag is negatively charged. Thus the separation of the cleaved electrophoretic tag from, not only uncleaved probe, but also its degradation products, is easily achieved by using conventional separation methods. Alternatively, the receptor may be bound to a solid support or high molecular weight macromolecule, such as a vessel wall, particles, e.g. magnetic particles, cellulose, agarose, etc., and separated by physical separation or centrifugation, dialysis, etc. This method further enhances the specificity of the assay and allows for a higher degree of multiplexing.

Fluorescent Quenching

A electrophoretic probe may have a combination of a quencher and a fluorescer attached so that the intact electrophoretic probe is prevented from fluorescing. The quencher and the fluorescer should be at different sides of the cleavage site. As the reaction proceeds and fluorescer is released from the probe and, therefore, removed from the quencher, it would then be capable of fluorescence. Uncleaved probe would be undetectable upon separation and would no interfer with the detection of released electrophoretic tags.

Synthesis of Probes

The chemistry for performing the types of syntheses to form the charge-imparting moiety or mobility modifier as a peptide chain is well known in the art. See, for example, Marglin, et al., Ann. Rev. Biochem. (1970) 39:841-866. In general, such syntheses involve blocking, with an appropriate protecting group, those functional groups that are not to be involved in the reaction. The free functional groups are then reacted to form the desired linkages. The peptide can be produced on a resin as in the Merrifield synthesis (Merrifield, J. Am. Chem. Soc. (1980) 85:2149-2154 and Houghten et al., Int. J. Pep. Prot. Res. (1980) 16:311-320. The peptide is then removed from the resin according to known techniques.

A summary of the many techniques available for the synthesis of peptides may be found in J. M. Stewart, et al., “Solid Phase Peptide Synthesis, W. H. Freeman Co, San Francisco (1969); and J. Meienhofer, “Hormonal Proteins and Peptides”, (1973), vol. 2, p. 46, Academic Press (New York), for solid phase peptide synthesis; and E. Schroder, et al., “The Peptides”, vol. 1, Academic Press (New York), 1965 for solution synthesis.

In general, these methods comprise the sequential addition of one or more amino acids, or suitably protected amino acids, to a growing peptide chain. Normally, a suitable protecting group protects either the amino or carboxyl group of the first amino acid. The protected or derivatized amino acid can then be either attached to an inert solid support or utilized in solution by adding the next amino acid in the sequence having the complementary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide. The protecting groups are removed, as desired, according to known methods depending on the particular protecting group utilized. For example, the protecting group may be removed by reduction with hydrogen and palladium on charcoal, sodium in liquid ammonia, etc.; hydrolysis with trifluoroacetic acid, hydrofluoric acid, and the like.

For synthesis of electrophoretic probes employing phosphoramidite, or related, chemistry many guides are available in the literature: Handbook of Molecular Probes and Research Products, 8^{th} edition (Molecular Probes, Inc., Eugene, Oreg., 2002); Beaucage and Iyer, Tetrahedron, 48: 2223-2311 (1992); Molko et al, U.S. Pat. No. 4,980,460; Koster et al, U.S. Pat. No. 4,725,677; Caruthers et al, U.S. Pat. Nos. 4,415,732; 4,458,066; and 4,973,679; and the like. Many of these chemistries allow components of the electrophoretic probe to be conveniently synthesized on an automated DNA synthesizer, e.g. an Applied Biosystems, Inc. (Foster City, Calif.) model 392 or 394 DNA/RNA Synthesizer, or the like.

Synthesis of e-tag reagents comprising nucleotides as part of the mobility-modifying moiety can be easily and effectively achieved via assembly on a solid phase support using standard phosphoramidite chemistries. The resulting mobility modifying moiety may be linked to the label and/or polypeptide-binding moiety as discussed above.

Synthesis of electrophoretic probes comprising nucleotides can be easily and effectively achieved via assembly on a solid phase support during probe synthesis, using standard phosphoramidite chemistries. The e-tag moieties are assembled at the 5′ end of probes after coupling of a final nucleosidic residue, which becomes part of the electrophoretic tag during the assay.

In one approach, the electrophoretic probe is constructed sequentially from a single or several monomeric phosphoramidite building blocks (one containing a dye residue), which are chosen to generate tags with unique electrophoretic mobilities based on their mass to charge ratio. The electrophoretic probe is thus composed of monomeric units of variable charge to mass ratios bridged by phosphate linkers.

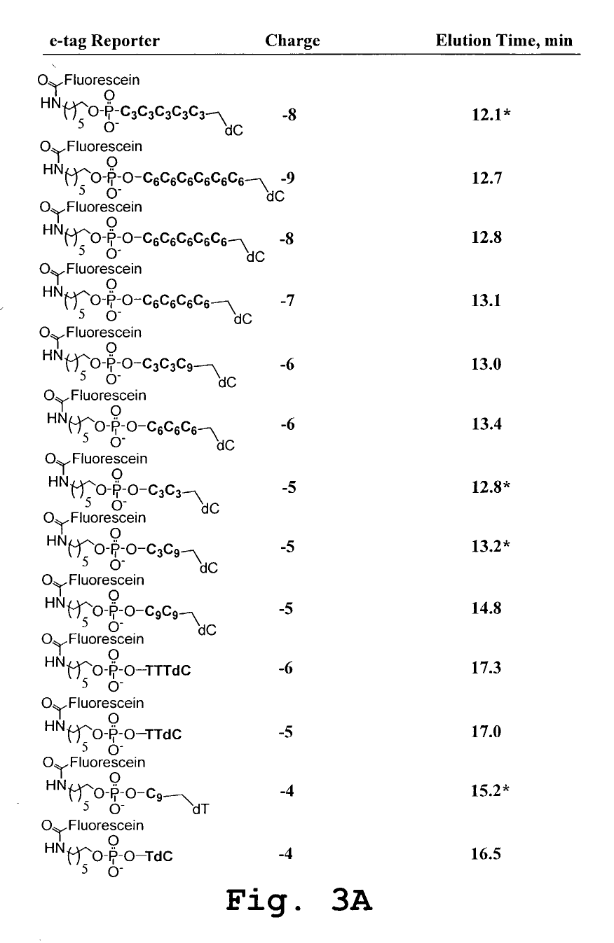

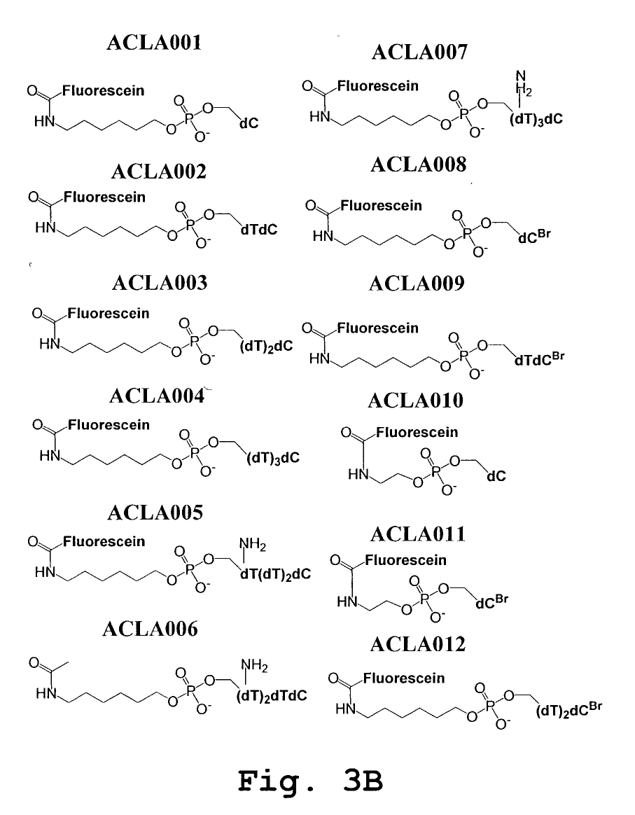

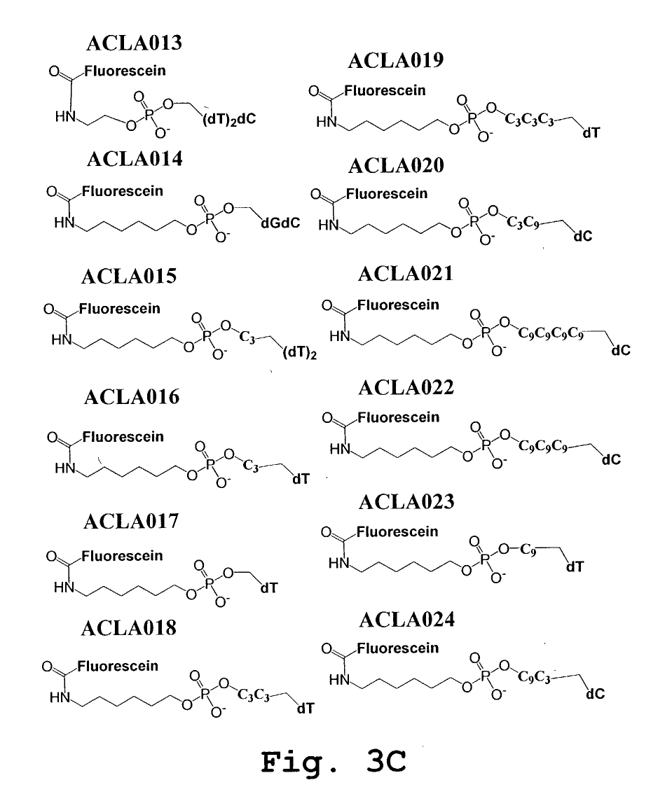

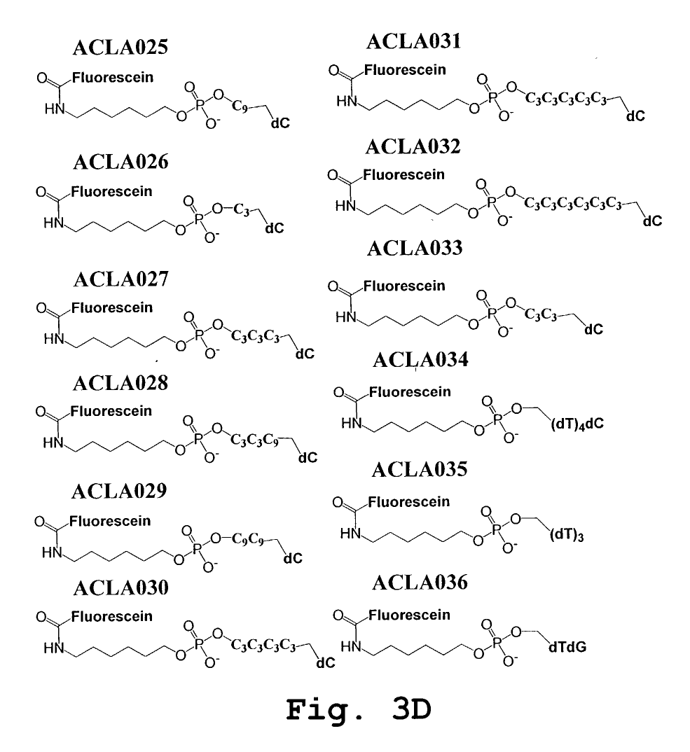

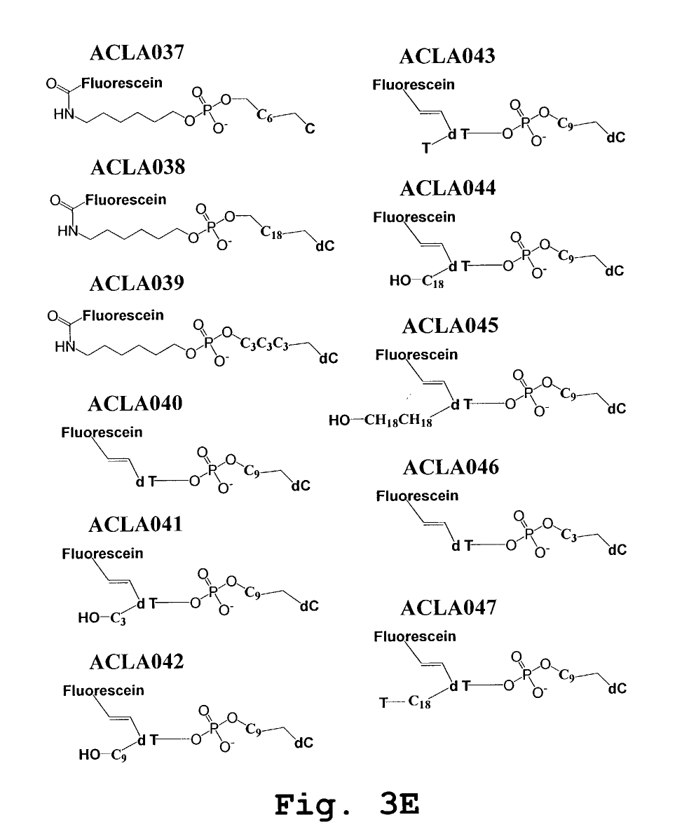

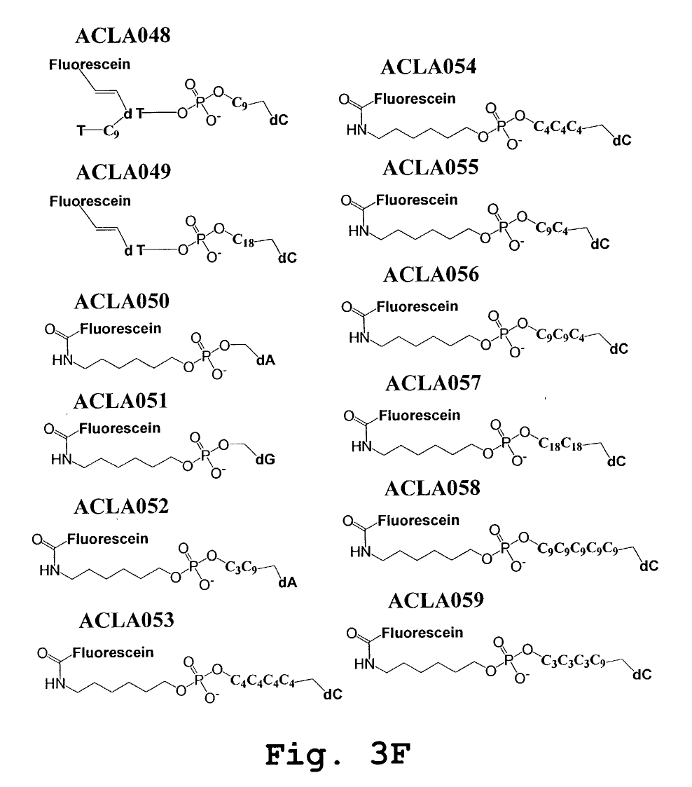

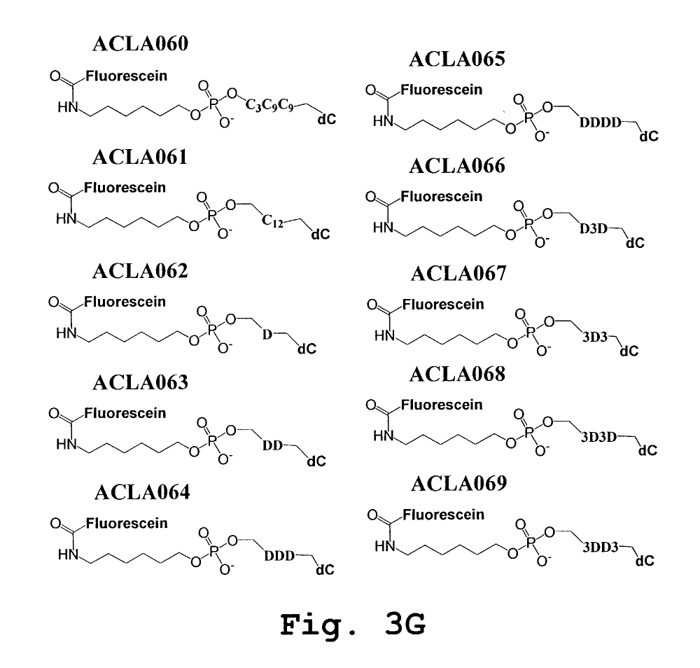

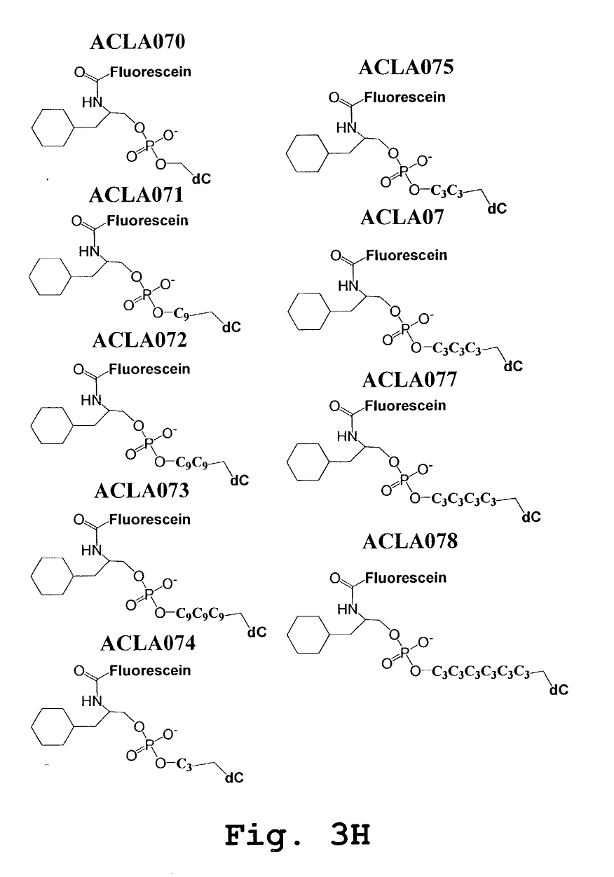

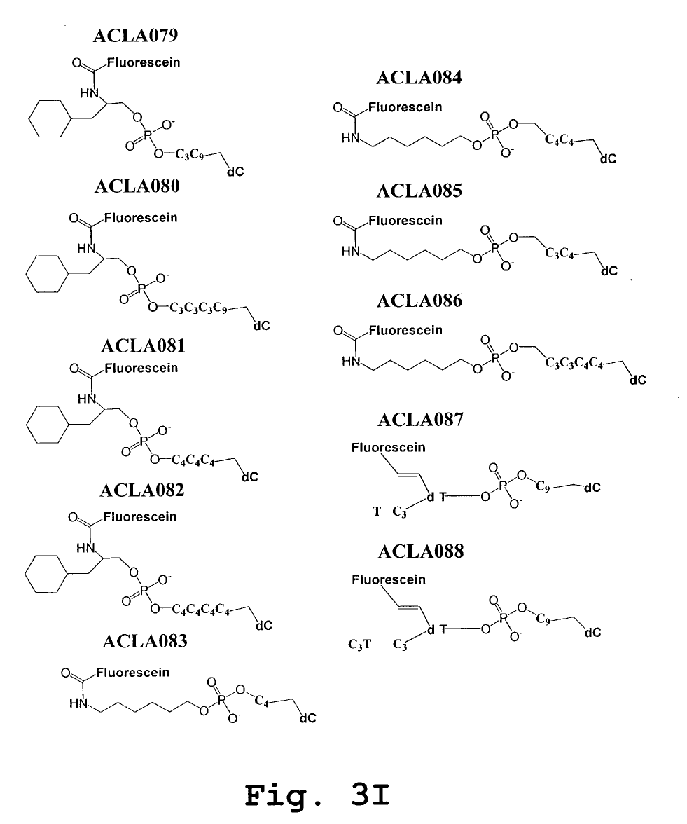

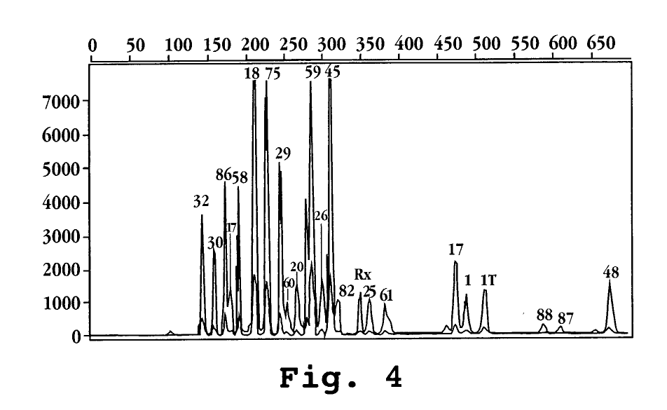

FIG. 3 illustrates predicted and experimental (*) elution times of electrophoretic tags. C₃, C₆, C₉, and C₁₈ are commercially available phosphoramidite spacers from Glen Research, Sterling Va. The units are derivatives of N,N-diisopropyl, O-cyanoethyl phosphoramidite, which is indicated by “Q”. C₃ is DMT (dimethoxytrityl)oxypropyl Q; C₆ is DMToxyhexyl Q; C₉ is DMToxy(triethyleneoxy) Q; C₁₂ is DMToxydodecyl Q; C₁₈ is DMToxy(hexaethyleneoxy) Q. E-tag moieties are synthesized to generate a contiguous spectrum of signals, one eluting after another with none of them coeluting (FIG. 4).

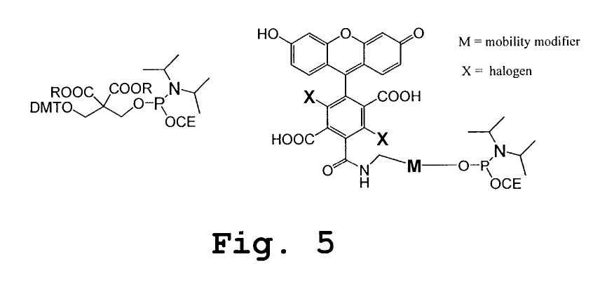

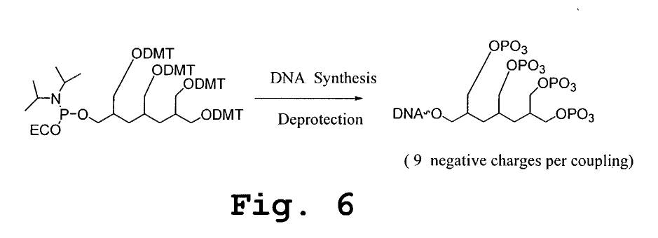

All of the above e-tag molecules work well and are easily separable and elute at 40 minutes. To generate tags that elute faster, highly charged low molecular weight tags are typically employed. Several types of phosphoramidite monomers allow for the synthesis of highly charged tags with early elution times. Use of dicarboxylate phosphoramidites (FIG. 5, left) allows for the addition of 3 negative charges per coupling of monomer. A variety of fluorescein derivatives (FIG. 5, right) allow the dye component of the tag to carry a higher mass than standard fluorescein. Polyhydroxylated phosphoramidites (FIG. 6) in combination with a common phosphorylation reagent enable the synthesis of highly phosphorylated tags. Combinations of these reagents with other mass modifier linker phosphoramidites allow for the synthesis of tags with early elution times.

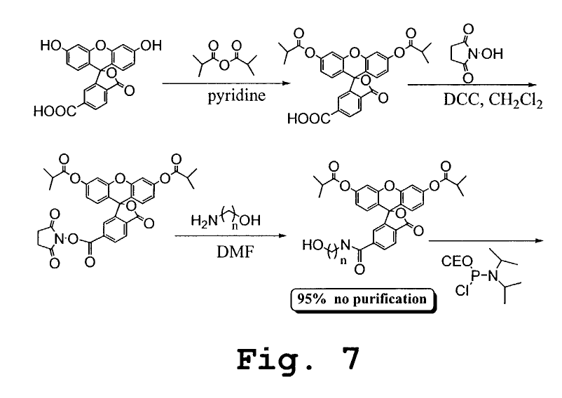

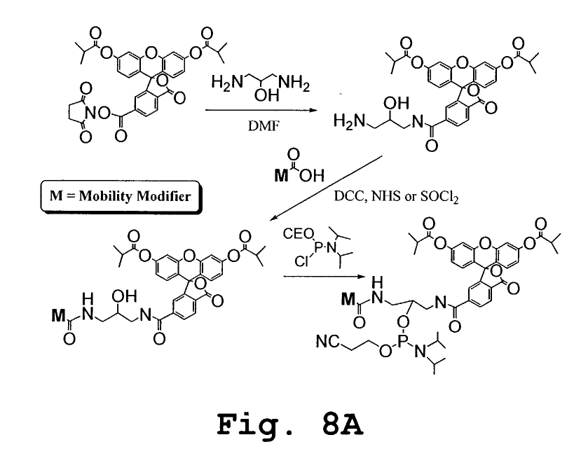

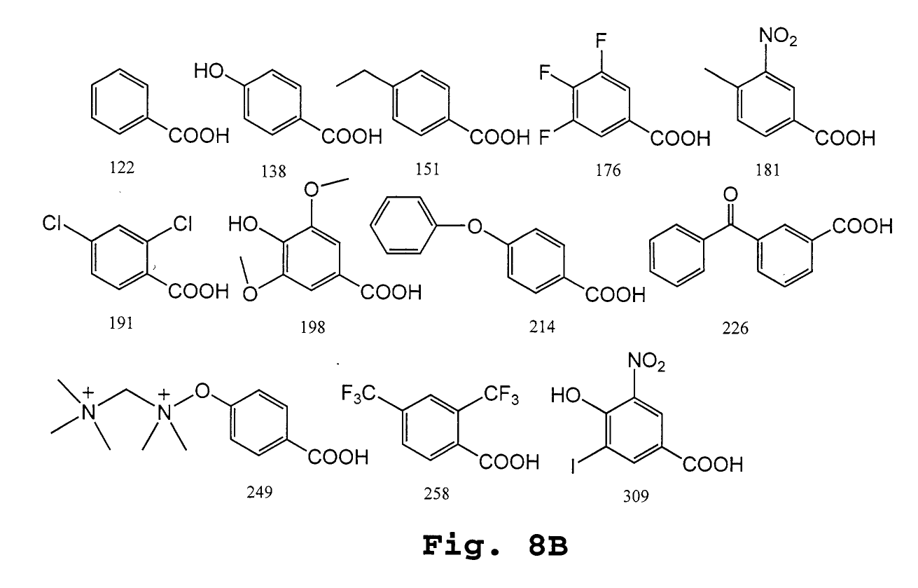

One exemplary synthetic approach is outlined in FIG. 7. Starting with commercially available 6-carboxy fluorescein, the phenolic hydroxyl groups are protected using an anhydride. Isobutyric anhydride in pyridine was employed but other variants are equally suitable. It is important to note the significance of choosing an ester functionality as the protecting group. This species remains intact though the phosphoramidite monomer synthesis as well as during oligonucleotide construction. These groups are not removed until the synthesized oligo is deprotected using ammonia. After protection the crude material is then activated in situ via formation of an N-hydroxy succinimide ester (NHS-ester) using DCC as a coupling agent. The DCU byproduct is filtered away and an amino alcohol is added. Many amino alcohols are commercially available some of which are derived from reduction of amino acids. Only the amine is reactive enough to displace N-hydroxy succinimide. Upon standard extractive workup, a 95% yield of product is obtained. This material is phosphitylated to generate the phosphoramidite monomer (FIG. 7). For the synthesis of additional e-tag moieties, a symmetrical bis-amino alcohol linker is used as the amino alcohol (FIG. 8A). As such, the second amine is then coupled with a multitude of carboxylic acid derivatives (exemplified by several possible benzoic acid derivatives shown in FIG. 8B) prior to the phosphitylation reaction. Using this methodology hundreds, even thousands of e-tag moieties with varying charge to mass ratios can easily be assembled during probe synthesis on a DNA synthesizer using standard chemistries.

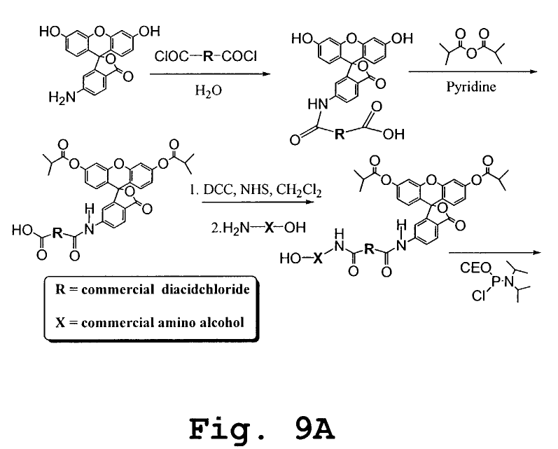

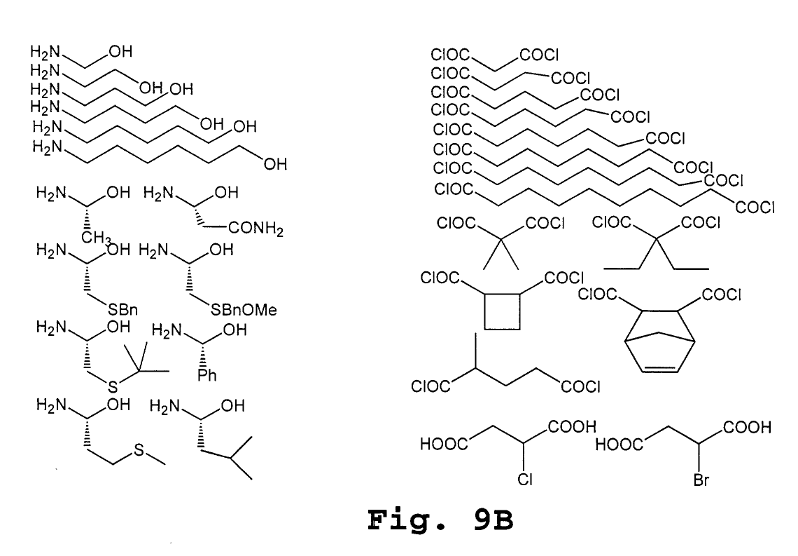

Alternatively, e-tag moieties are accessed via an alternative strategy that uses 5-aminofluorescein as starting material (FIG. 9A). Addition of 5-aminofluorescein to a great excess of a di-acid dichloride in a large volume of solvent allows for the predominant formation of the monoacylated product over dimer formation. The phenolic groups are not reactive under these conditions. Aqueous workup converts the terminal acid chloride to a carboxylic acid. This product is analogous to 6-carboxyfluorescein, and using the same series of steps is converted to its protected phosphoramidite monomer (FIG. 9A). There are many commercially available diacid dichorides and diacids, which can be converted to diacid dichlorides using SOCl₂ or acetyl chloride. This methodology is highly attractive in that a second mobility modifier is used. As such, if one has access to 10 commercial modified phosphoramidites and 10 diacid dichlorides and 10 amino alcohols there is a potential for 1000 different e-tag moieties. There are many commercial diacid dichlorides and amino alcohols (FIG. 9B). These synthetic approaches are ideally suited for combinatorial chemistry.

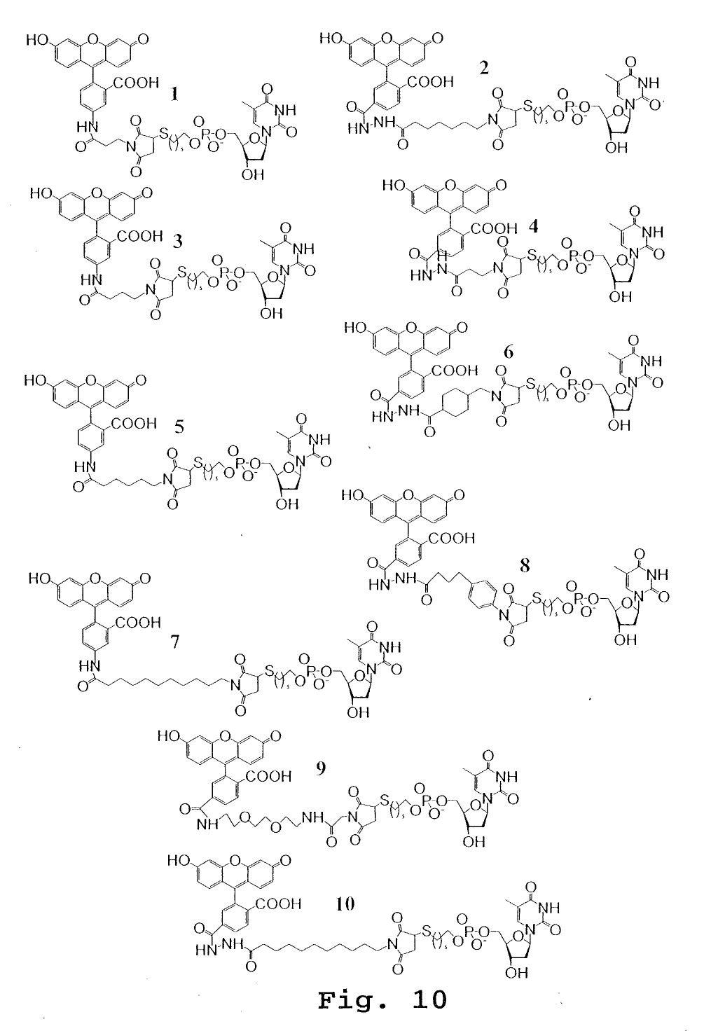

A variety of maleimide-derivatized e-tag moieties have also been synthesized. These compounds were subsequently bioconjugated to 5′-thiol derivatized DNA sequences and subjected to the 5′-nuclease assay. Exemplary species formed upon cleavage are depicted in FIG. 10.

The electrophoretic tag may be assembled having an appropriate functionality at one end for linking to the binding compound. Thus for oligonucleotides, one would have a phosphoramidite or phosphate ester at the linking site to bond to an oligonucleotide chain, either 5′ or 3′, particularly after the oligonucleotide has been synthesized, while still on a solid support and before the blocking groups have been removed. While other techniques exist for linking the oligonucleotide to the electrophoretic tag, such as having a S functionality at the oligonucleotide terminus that specifically reacts with a functionality on the electrophoretic tag, such as maleimide and thiol, or amino and carboxy, or amino and keto under reductive amination conditions, the phosphoramidite addition is preferred.

Of particular interest in preparing electrophoretic probes is using the solid support phosphoramidite chemistry to build the electrophoretic probe as part of the oligonucleotide synthesis. Using this procedure, one attaches the next succeeding phosphate at the 5′ or 3′ position, usually the 5′ position of the oligonucleotide chain. The added phosphoramidite may have a natural nucleotide or an unnatural nucleotide. Instead of phosphoramidite chemistry, one may use other types of linkers, such as thio analogs, amino acid analogs, etc. Also, one may use substituted nucleotides, where the mass-modifying region and/or the charge-modifying region may be attached to the nucleotide, or a ligand may be attached to the nucleotide. In this way, phosphoramidite links are added comprising the regions of the electrophoretic probe, whereby when the synthesis of the oligonucleotide chain is completed, one continues the addition of the regions of the electrophoretic tag to complete the molecule. Conveniently, one would provide each of the building blocks of the different regions with a phosphoramidite or phosphate ester at one end and a blocked functionality, where the free functionality can react with a phosphoramidite, mainly a hydroxyl. By using molecules for the different regions that have a phosphoramidite at one site and a protected hydroxyl at another site, the electrophoretic probe can be built up until the terminal region, which does not require the protected hydroxyl.

Illustrative of the synthesis would be to employ a diol, such as an alkylene diol, polyalkylene diol, with alkylene of from two to three carbon atoms, alkylene amine or poly(alkylene amine) diol, where the alkylenes are of from two to three carbon atoms and the nitrogens are substituted, for example with blocking groups or alkyl groups of from one to six carbon atoms, where one diol is blocked with a conventional protecting group, such as a dimethyltrityl group. This group can serve as the mass-modifying region and with the amino groups as the charge-modifying region as well. If desired, the mass modifier can be assembled using building blocks that are joined through phosphoramidite chemistry. In this way the charge modifier can be interspersed between within the mass modifier. For example, one could prepare a series of polyethylene oxide molecules having 1, 2, 3 . . . n units. Where one wished to introduce a number of negative charges, one could use a small polyethylene oxide unit and build up the mass and charge-modifying region by having a plurality of the polyethylene oxide units joined by phosphate units. Alternatively, by employing a large spacer, fewer phosphate groups would be present, so that without large mass differences, one would have large differences in mass-to-charge ratios.

The chemistry that is employed is the conventional chemistry used in oligonucleotide synthesis, where building blocks other than nucleotides are used, but the reaction is the conventional phosphoramidite chemistry and the blocking group is the conventional dimethoxyltrityl group. Of course, other chemistries compatible with automated synthesizers can also be used, but there is no reason to add additional complexity to the process.

Separation and Detection

Electrophoretic tags may be designed to be separated by a variety liquid phase separation techniques, including electrophoresis and chromatography. Preferably, the separation technique selected provides as a data readout a separation profile, such as an electropherogram or a chromatograph, where electrophoretic tags of a plurality being used are distinguishable as separate peaks or bands. The composition of the mobility modifying region and detectable label is selected with respect to the separation technique being employed. Preferably, released electrohoretic tags are separated electrophoretically.

Methods for electrophoresis of are well known and are described, for example, in Krylov et al, Anal. Chem., 72: 111R-128R (2000); P. D. Grossman and J. C. Colburn, Capillary Electrophoresis: Theory and Practice, Academic Press, Inc., NY (1992); U.S. Pat. Nos. 5,374,527; 5,624,800; 5,552,028; ABI PRISM 377 DNA Sequencer User's Manual, Rev. A, January 1995, Chapter 2 (Applied Biosystems, Foster City, Calif.); and the like. A variety of suitable electrophoresis media are commercially available from Applied Biosystems and other vendors, including non-crosslinked media, for use with automated instruments such as the Applied Biosysterns “3700” and“3100”Instruments, for example. Optimal electrophoresis conditions, e.g., polymer concentration, pH, temperature, voltage, concentration of denaturing agent, employed in a particular separation depends on many factors, including the size range of the compounds to be separated, their compositions, and the like. Accordingly application of the invention may require standard preliminary testing to optimize conditions for particular separations.

During or after electrophoretic separation, the electrophoretic tags are detected or identified by recording fluorescence signals and migration times (or migration distances) of the separated compounds, or by constructing a chart of relative fluorescent and order of migration of the electrophoretic tags (e.g., as an electropherogram). To perform such detection, the electrophoretic tags can be illuminated by standard means, e.g. a high intensity mercury vapor lamp, a laser, or the like. Typically, the electrophoretic tags are illuminated by laser light generated by a He—Ne gas laser or a solid-state diode laser. The fluorescence signals can then be detected by a light-sensitive detector, e.g., a photomultiplier tube, a charged-coupled device, or the like. Exemplary electrophoresis detection systems are described elsewhere, e.g., U.S. Pat. Nos. 5,543,026; 5,274,240; 4,879,012; 5,091,652; 6,142,162; or the like.

After completion of the reaction, which may be monitored, for example, by monitoring the change in signal such as, e.g., fluorescence as described above, or taking aliquots and assaying for total free electrophoretic tags, the mixture may now be analyzed. Depending on the instrument, from one to four different fluorescers activated by the same light source and emitting at different detectable labels may be used. With improvements, five or more different fluorescers may be available, where an additional light source may be required. Electrochemical detection is described in U.S. Pat. No. 6,045,676.

In one embodiment of the presence of each of the cleaved electrophoretic tags is determined by the fluorescent label contained in the electrophoretic tag. The separation of the mixture of labeled electrophoretic tags is typically carried out by electroseparation, which involves the separation of components in a liquid by application of an electric field, preferably, by electrokinesis (electrokinetic flow) or electrophoretic flow, or a combination of electrophoretic flow within electroosmotic flow, with the separation of the electrophoretic tag mixture into individual fractions or bands. Electroseparation involves the migration and separation of molecules in an electric field based on differences in mobility. Various forms of electroseparation include, by way of example and not limitation, free zone electrophoresis, gel electrophoresis, isoelectric focusing, isotachophoresis, capillary electrochromatography, and micellar electrokinetic chromatography. Capillary electrophoresis involves electroseparation, preferably by electrokinetic flow, including electrophoretic, dielectrophoretic and/or electroosmotic flow, conducted in a tube or channel of about 1 to about 200 micrometer, usually, about 10 to about 100 micrometers cross-sectional dimensions. The capillary may be a long independent capillary tube or a channel in a wafer or film comprised of silicon, quartz, glass or plastic.

In capillary electroseparation, an aliquot of the reaction mixture containing the electrophoretic tags is subjected to electroseparation by introducing the aliquot into an electroseparation channel that may be part of, or linked to, a capillary device in which the amplification and other reactions are performed. An electric potential is then applied to the electrically conductive medium contained within the channel to effectuate migration of the components within the combination. Generally, the electric potential applied is sufficient to achieve electroseparation of the desired components according to practices well known in the art. One skilled in the art will be capable of determining the suitable electric potentials for a given set of reagents used in the present invention and/or the nature of the cleaved labels, the nature of the reaction medium and so forth. The parameters for the electroseparation including those for the medium and the electric potential are usually optimized to achieve maximum separation of the desired components. This may be achieved empirically and is well within the purview of the skilled artisan.

For a homogeneous assay, the sample, the first and electrophoretic probes, and ancillary reagents are combined in a reaction mixture supporting the cleavage of the linking region. The mixture may be processed to separate the electrophoretic tags from the other components of the mixture. The mixture, with or without electrophoretic tag enrichment, may then be transferred to an electrophoresis device, usually a microfluidic or capillary electrophoresis device and the medium modified as required for the electrophoretic separation. Where one wishes to remove from the separation channel intact electrophoretic tag molecules, a ligand is bound to the electrophoretic tag that is not released when the electrophoretic tag is released. Alternatively, by adding a reciprocal binding member that has the opposite charge of the electrophoretic tag, so that the overall charge is opposite to the charge of the electrophoretic tag, these molecules will migrate toward the opposite electrode from the released electrophoretic tag molecules. For example, one could use biotin and streptavidin, where streptavidin carries a positive charge. In the case of a peptide analyte, one embodiment would have cleavage at a site where the ligand remains with the peptide analyte. For example, one could have the electrophoretic tag substituted for the methyl group of methionine. Using the pyrazolone of the modified methionine, one could bond to an available lysine. The amino group of the pyrazolone would be substituted with biotin. Cleavage would then be achieved with cyanogen bromide, releasing the electrophoretic tag, but the biotin would remain with the peptide and any electrophoretic tag that was not released from the binding member. Avidin is then used to change the polarity or sequester the electrophoretic tag conjugated to the target-binding moiety for the analyte or target-binding moiety.

For capillary electrophoresis one may employ one or more detection zones to detect the separated cleaved labels. It is, of course, within the purview of the present invention to utilize several detection zones depending on the nature of the reactions, mobility-modifying moieties, and so forth. There may be any number of detection zones associated with a single channel or with multiple channels. Suitable detectors for use in the detection zones include, by way of example, photomultiplier tubes, photodiodes, photodiode arrays, avalanche photodiodes, linear and array charge coupled device (CCD) chips, CCD camera modules, spectrofluorometers, and the like. Excitation sources include, for example, filtered lamps, LEDs, laser diodes, gas, liquid and solid-state lasers, and so forth. The detection may be laser scanned excitation, CCD camera detection, coaxial fiber optics, confocal back or forward fluorescence detection in single or array configurations, and the like.

Detection may be by any of the known methods associated with the analysis of capillary electrophoresis columns including the methods shown in U.S. Pat. Nos. 5,560,811 (column 11, lines 19-30), U.S. Pat. Nos. 4,675,300, 4,274,240 and 5,324,401, the relevant disclosures of which are incorporated herein by reference. Those skilled in the electrophoresis arts will recognize a wide range of electric potentials or field strengths may be used, for example, fields of 10 to 1000 V/cm are used with about 200 to about 600 V/cm being more typical. The upper voltage limit for commercial systems is about 30 kV, with a capillary length of about 40 to about 60 cm, giving a maximum field of about 600 V/cm. For DNA, typically the capillary is coated to reduce electroosmotic flow, and the injection end of the capillary is maintained at a negative potential.

For ease of detection, the entire apparatus may be fabricated from a plastic material that is optically transparent, which generally allows light of wavelengths ranging from about 180 to about 1500 nm, usually about 220 to about 800 nm, more usually about 450 to about 700 nm, to have low transmission losses. Suitable materials include fused silica, plastics, quartz, glass, and so forth.

Kits

Another aspect of the present invention provides a kit comprising a probe capable of forming a recognition structure upon binding to the target sequence, and an enzyme that can be used to cleave the structure. For an indirect method, the probe is a pair of probes in accordance with the present invention.

For multiplex reactions, the present invention also provides a kit comprising a set of probes that can be used to detect or quantitate a plurality of target sequences in parallel. Again, for an indirect method, the probe is a pair of probes in accordance with the present invention. The kit may further comprise an enzyme capable of recognizing and cleaving these probes upon binding of each probe to its corresponding target sequence.

Other methods and kits of the present invention can be formulated by a person of ordinary skills in the art according to the present disclosure. The following examples are offered to illustrate this invention and are not to be construed in any way as limiting the scope of the present invention.

### EXAMPLE 1

### Target Recognition Using a Restriction Enzyme in a Two-Probe Assay

20 μl reactions were assembled containing:

2 μl of 10× restriction enzyme reaction buffer (New England BioLabs, Beverly, Mass.)

0.2 μl of 10 μg/μl acetylated BSA (New England BioLabs)

1 μl of 20 μM activation probe

1 μl of 20 μM signal probe

0.5 μl of 10 U/μl TaqI restriction enzyme (New England BioLabs)

Target DNA

Nuclease free water (Ambion Inc., Mass.), to bring to a final volume of 20 μl

The reaction mix was incubated at 60° C. for 4 hours. 10 μl of the reaction product was mixed with 1 μl of 100 nM fluorescein to serve as an internal standard and 1 μl of 10 mg/ml avidin (Sigma, St. Louise, Mo.) in a PE optical plate. The products were separated using ABI 3100 genetic analyzer (PE Corp.). The running conditions were set as: run temperature of 30° C., pre run voltage of 15 kV, pre run time of 180 seconds, injection voltage of 3 KV, injection time of 100 seconds, run voltage of 15 KV, run time of 1200 seconds and sampling rate of 140 data points per msec.

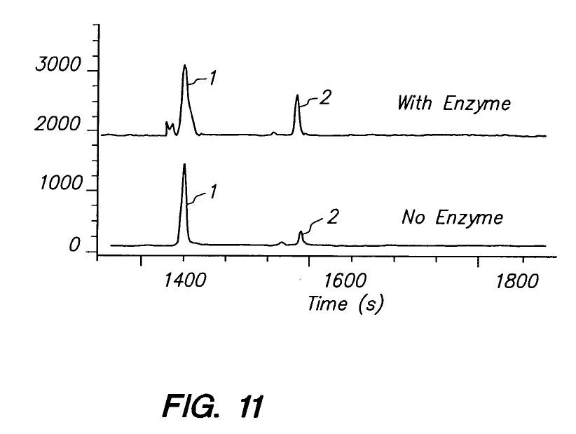

Exemplary results are shown in FIG. 11. The upper panel shows the products of a reaction containing restriction enzyme. The internal control (FAM) eluted at around 1400 seconds (peak 1). The released electrophoretic tag eluted at around 1550 seconds (peak 2). The lower panel shows a control reaction performed without restriction enzyme. The unlabeled peaks in these electropherograms, predominantly to the left of peak 1, are nonspecific reaction products or contaminants from the probe preparation. The probes and synthetic target sequences are listed below, all given in the 5′ to 3′ direction:

Helper probe:

CCTTCCTTATCCTGGATCTTGGCAAAATCGA (SEQ ID NO: 1)

Electrophoretic probe:

Tag1-TCGATTTTCTTTACATTTTCTATCGTATCCG-biotin (SEQ ID NO: 2)

Synthetic oligonucleotide target:

(SEQ ID NO:3)

GTAAAAACCCTTACGGGGAAGACCATCACCCTCGAGGTTGAACCCTCGGA
TACGATAGAAAATGTAAAGGCCAAGATCCAGGATAAGGAAGGAATTCCTC
CTGATCAGCAGAGACTGATTTGCTGGCAAGCAGCTGGAAGATGGACGT
ACTTTGTCTGACTACAA

The recognition sequence for TaqI is TCGA, which is underlined in the sequence of the probes. Cleavage occurs after the thymidine residue, releasing Tag1-T as the electrophoretic tag. The target DNA was either 1 μl of an in vitro transcript of the ubiquitin gene at a concentration of 10⁹ copies/μl, or 200 pM of synthetic oligonucleotide.

### EXAMPLE 2

### Target Recognition Using a DNA Repair Enzyme in a Two-Probe Assay

DNA repair enzymes can also be utilized in practicing the invention. For example, assay similar to that described above, but utilizing human apurinic/apyrimidinic endonuclease (APE), may be assembled with the following components:

2 μl of 10×APE reaction buffer (Trevigen, Gaithersburg, Md.)

1 μl of 20 μM activation probe

1 μl of 20 μM signal probe

10 μl of 0.1 U/μl APE enzyme (Trevigen, Gaithersburg, Md.),

1 μl of target DNA

Nuclease free water (Ambion Inc., Mass.) will be added to a final volume of 20 μl

The reaction mix should be incubated at conditions appropriate for the chosen enzyme activity, e.g., 37° C. for 4 hours in this example. The products may be resolved via capillary electrophoresis, as described above, using the same instrument and running conditions. The recognition sequence for APE is the abasic site, underlined in the signal probe sequence, with cleavage occurring at the 3′-end of the ribose. Cleavage of the signal probe will release an electrophoretic tag with the expected mobility of a molecule of the composition Tag1-Cds(C).

The probes and synthetic target sequences are listed below, all given in the 5′ to 3′ direction:

Helper probe:

ATCCTGGATCTTGGCAAGGAGGGGAACTGATCCCCT (SEQ ID NO: 4)

Electrophoretic probe:

Tag1-Cds(C)TTCTTTACATTTTCTAT-biotin (SEQ ID NO: 5), where ds(C) is the abasic site;

Synthetic oligonucleotide target:

(SEQ ID NO:3)

GTAAAAACCCTTACGGGGAAGACCATCACCCTCGAGGTTGAACCCTCGGA
TACGATAGAAAATGTAAAGGCCAAGATCCAGGATAAGGAAGGAATTCCTC
CTGATCAGCAGAGACTGATCTTTGCTGGCAAGCAGCTGGAAGATGGACGT
ACTTTGTCTGACTACAA

The target DNA was either an in vitro transcript of the ubiquitin gene at a concentration of 10⁹ copies/μl, or 200 pM of synthetic oligonucleotide.

We claim:

1. A method of detecting a plurality of polynucleotides in a sample, the method comprising the steps of:
providing for each polynucleotide a helper probe complementary to a region of the polynucleotide and an electrophoretic probe complementary to the helper probe and to the polynucleotide adjacent to said region, such that the helper probe and the electrophoretic probe form a recognition duplex upon hybridization to each other and to the polynucleotide, each electrophoretic probe having attached an electrophoretic tag with a separation or detection characteristic distinct from those of other electrophoretic tags so that each electrophoretic tag forms a distinguishable peak in a separation profile;
combining under hybridization conditions the sample, the helper probes, and the electrophoretic probes to form an assay mixture such that recognition duplexes are formed;
cleaving the recognition duplexes at a cleavage site so that electrophoretic tags are released; and
separating and identifying the released electrophoretic tags to detect each of the plurality of polynucleotides.

2. The method of claim 1 wherein each of said released electrophoretic tags has a molecular weight of from 150 to 5000 daltons.

3. The method of claim 2 including, prior to said step of separating, a further step of treating said assay mixture to exclude from said separation profile uncleaved electrophoretic probes.

4. The method of claim 3 wherein each of said electrophoretic probes has a capture ligand attached to a nucleotide located opposite said cleavage site from said electrophoretic tag and wherein said step of treating further includes reacting the capture ligand with a capture agent.

5. The method of claim 3 wherein said step of cleaving produces a released electrophoretic tag having a charge opposite that of said uncleaved electrophoretic probe.

6. The method of claim 3 wherein each of said electrophoretic probes has a quencher attached to a nucleotide located opposite said cleavage site from said electrophoretic tag such that upon separating uncleaved electrophoretic probes generate no signal in said separation profile.

7. The method in accordance with any of claims 1, 2, 3, 4, 5, or 6 wherein said said separation characteristic is electrophoretic mobility and wherein said plurality is in the range from 5 to 100.

8. The method of claim 7 wherein each of said released electrophoretic tags has a distinct charge/mass ratio in the range of from −0.001 to 0.5.

9. The method of claim 7 wherein at least one of said released electrophoretic tags has a positive charge.

10. The method of claim 7 wherein every said released electrophoretic tag has a negative charge.

11. The method of claim 7 wherein said step of cleaving is carried out by a hOGG1 protein and said electrophoretic probe is defined by the formula:
3′—(N)ⱼ—Z—(B)ₖ—(M,D)
wherein B and N are each a nucleotide; j is an integer in the range of from 8 to 40; k is an integer in the range of from 1 to 3; Z is selected from the group consisting of 7,8-dihydro-8-oxo-2′-deoxyguanosine, foramidopyrimidine guanosine, and methylforamidopyrimidine guanosine; D is a fluorescent dye; and M is a mobility modifying moiety that is a bond or an organic molecule having up to 100 atoms other than hydrogen selected from the group consisting of carbon, oxygen, nitrogen, phosphorus, boron, and sulfur.

12. The method of claim 11 wherein Z is 7,8-dihydro-8-oxo-2′-deoxyguanosine and wherein at least one N has said capture ligand attached.

13. A kit for detecting the presence or absence of one or more target polynucleotides in a sample, the kit comprising:
a plurality of pairs of helper probes and electrophoretic probes, each helper probe of a pair being complementary to a region of a target polynucleotide and each electrophoretic probe of the same pair being complementary to the helper probe and to the target polynucleotide adjacent to said region, such that the helper probe and the electrophoretic probe form a recognition duplex upon hybridization to each other and to the target polynucleotide, each electrophoretic probe having attached an electrophoretic tag with a separation or detection characteristic distinct from those of other electrophoretic tags so that each electrophoretic tag forms a distinguishable peak in a separation profile.

14. The kit of claim 13 further including a cleavage agent for recognizing and cleaving said recognition duplex.

15. The kit of claim 13 wherein said electrophoretic probe is selected from a group defined by the formula:
(D,M)—T
wherein D is a fluorescent dye; M is a mobility modifying moiety that is a bond or an organic molecule having up to 100 atoms other than hydrogen selected from the group consisting of carbon, oxygen, nitrogen, phosphorus, boron, and sulfur; and T is an oligonucleotide having a capture ligand attached.

16. The kit of claim 15 wherein said electrophoretic probe is selected from the group defined by the formula:
3′—(N)ⱼ—Z—(B)ₖ—(M,D)
wherein B and N are each a nucleotide; j is an integer in the range of from 8 to 40; k is an integer in the range of from 1 to 3; and Z is selected from the group consisting of 7,8-dihydro-8-oxo-2′-deoxyguanosine, foramidopyrimidine guanosine, and methylforamidopyrimidine guanosine.

17. The kit in accordance with claims 13, 14, 15, or 16 wherein said plurality of pairs of probes is in the range of from 5 to 50.
